(19) United States
(12) Patent Application Publication
(10) Pub. No.: US 2012/0035224 A1
(43) Pub. Date: Feb. 9, 2012

(54) Betulin derived compounds as anti-feedants for plant pests

(75) Inventors: Sami Alakurtti, Vantaa (FI); Jari Yli-Kauhaluoma, Helsinki (FI); Taru Mäkelä, Helsinki (FI); Salem Koskimies, Helsinki (FI); Pia Bergström, Kirkkonummi (FI); Heikki Hokkanen, Espoo (FI); Ingeborg Menzler-Hokkanen, Espoo (FI)

(73) Assignee: Valtion teknillinen tutkimuskeskus, Espoo (FI)

(21) Appl. No.: 12/308,056

(22) PCT Filed: Jun. 1, 2007
(86) PCT No.: PCT/FI2007/050318

§ 371 Date: Jul. 6, 2011

(30) Foreign Application Priority Data
Jun. 7, 2006 (FI) 20065389

(51) Int. Cl. A01N 37/02; C07C 69/708; C07C 69/736; C07C 69/757; A01P 17/00; C07C 69/618; C07C 69/013; C07D 213/80; A01N 43/40; A01N 43/08; C07C 69/604; C07C 69/743

(52) U.S. Cl. 514/356; 560/181; 560/126; 560/61; 560/60; 560/6; 560/124; 560/104; 554/228; 554/223; 546/322; 514/510; 514/473

(57) ABSTRACT

The invention relates to compounds derived from betulin, and to the use thereof in plant pest control, particularly as antifeedants for butterfly larvae, beetles and snails. Further, the invention relates to novel betulin derivatives and methods for the production thereof either directly from betulin, or via intermediates derived therefrom.

### FIELD OF THE INVENTION

The invention relates to compounds derived from betulin, and to the use thereof in plant pest control, particularly as antifeedants for butterfly larvae, beetles and snails. Further, the invention relates to novel betulin derivatives and methods for the production thereof either directly from betulin, or from intermediates derived thereof.

### PRIOR ART

Betulin having the structure 1 shown below is a naturally occuring pentacyclic triterpene alcohol of the lupane family, also known as betulinol and lup-20(29)-ene-3β,28-diol. Betulin is found in the bark of some tree species, particularly in the birch (Betula sp.) bark at best in amounts up to 40% of the bark dry weight. In addition to betulin, also minor amounts of betulin derivatives are obtained from tree bark. There are known methods mainly based on extraction, for the isolation of betulin from bark material.

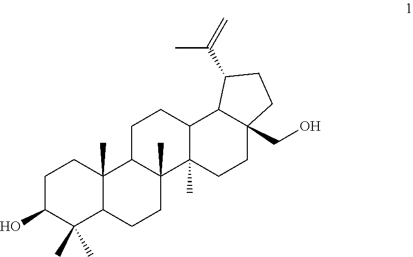

In some applications, poor solubility of betulin causes problems with respect to use and formulation, and accordingly, betulin is converted to its derivatives to improve the solubility. In the production of said derivatives, the reactivities of the functional groups of betulin, that is, the primary and secondary hydroxyl groups and the double bond are typically utilized. Both hydroxyl groups may be esterified, thus obtaining mono- or diesters. Also glycoside derivatives may be produced from betulin using known procedures, and betulin may be subjected to oxidation, reduction and rearrangement reactions in the presence of suitable oxidating or reducing agent or an acid catalyst.

Betulinic acid having the structure 3 shown in the reaction scheme below may be isolated e.g. from birch (Betula sp.) bark or cork of cork oak (Quercus suber L.) by extraction, and further, it may be produced by several methods mainly based on direct oxidation of the betulin or birch bark material. The reaction scheme shows the direct oxidation of betulin 1 according to U.S. Pat. No. 6,280,778 as Jones oxidation in the presence of a chromium(VI) oxide catalyst to give betulonic acid 2, followed by the selective reduction of the betulonic acid 2 thus obtained with sodium borohydride to give betulinic acid 3.

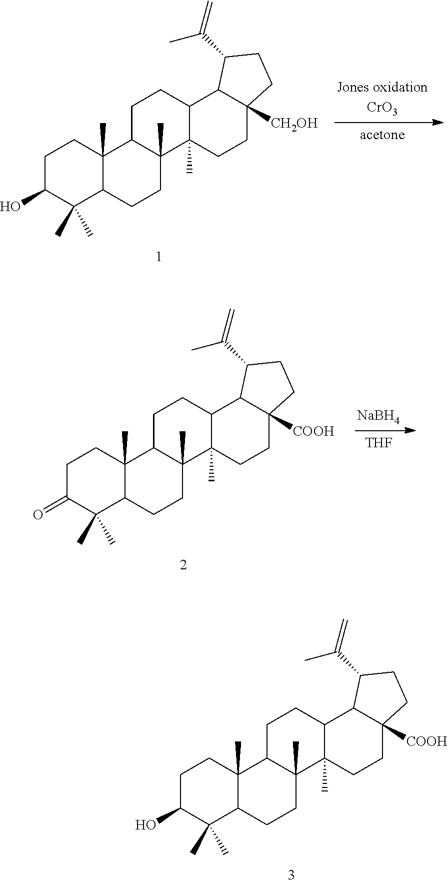

An alternative process for the production of betulinic acid is disclosed in patent U.S. Pat. No. 5,804,575, comprising an oxidation step where the 3-beta-hydroxyl of betulin is protected by acetylation. Isomerization and oxidation of the secondary hydroxyl group of betulin is thus prevented.

Use of betulin and derivatives thereof in agricultural and industrial chemical applications has been widely studied. For betulin and several derivatives thereof, activity against some bacteria and viruses and antifungal activity is found.

Pentacyclic triterpenoids are suggested for agricultural applications, particularly for plant protection applications to control microorganisms pathogenic for plants. The document WO2000033846 discloses the use of betulin, mono- and disuccinates, and glutarates thereof in fungicidal applications.

J. Agric. Food Chem. 1995, 43, 2513-2516 discloses the use of some ester derivatives of betulin, particularly betulin 20,29-epoxy-3β,28-diacetate, 30-hydroxylup-20(29)-ene-3β,28-diacetate and 30-hydroxy-20-oxo-29-norlup-3β,28-diacetate as an antifeedant agent for the control of Colorado beetles. An agent having activity against Colorado beetles with a dosage of ED₅₀=8 μg/cm², said activity being comparable to that of limonene, was obtained by an oxidative and rather complex modification of the betulin diacetate side chain. On the contrary, the compounds had no activity against the moth Helicoverpa zea, a cotton pest used as the other test insect.

J. Agric. Food Chem. 1998, 46, 2797-2799 describes the antifeedant activity of some betulin derivatives on tobacco worm (Spodoptera litura, Fabritius: subfamily Amphipyrinae of the family Noctuidae). Betulin derivatives containing a methylcinnamic acid, methyloxycinnamic acid or tri-O-methylgallate groupadded to the C-3 position were found as the most effective compounds.

Antifeedants of pests are generally very specific with regard to the activity, and synergistic effect with conventional synthetic pest control agents are found for some of them. Insects, particularly different beetle species typically have very different ways of life and preferences. Accordingly, an agent active against one species may not have any effect on some other species.

In the future, the use of antifeedants for plant pests will be a strategically significant alternative to traditional pesticides. Products found in the nature and their semisynthetic analogs and derivatives are primarily seeked to be utilized to attain a mechanism of activity and biodegrability as predictable as possible. Such products are, however, hardly used since there are only few suitable compounds that may be isolated from nature by feasible processes and in high amounts in pure form.

On the basis of above teachings, it may be seen that there is an obvious need for to environmentally acceptable compounds having desired efficient antifeedant activities on pests.

Compounds derived from betulin refer here to pentacyclic triterpenoids, particularly to betulin, betulinic and betulonic acids and derivatives thereof comprising natural compounds and/or compounds with known low toxicity as substituents, and especially to alcohol, phenol and/or carboxylic acid and/or ester and/or amide and/or ether derivatives of betulin.

Here, antifeedant agents refer to agents that inhibit feeding of pests.

### OBJECTS OF THE INVENTION

An object of the invention is the use of compounds derived from betulin as antifeedant agents for plant pests.

Another object of the invention is the use of compounds derived from betulin as antifeedant agents for butterflies, beetles and snails.

Still another object of the invention is to provide novel betulin derivatives.

Yet another object of the invention is to provide methods for producing said novel betulin derivatives, either directly from betulin, or from intermediates derived from betulin.

Characteristic features of said compounds derived from betulin, betulin derivatives, and methods for the production thereof, according to the invention, are disclosed in the claims.

### GENERAL DESCRIPTION OF THE INVENTION

The present invention is directed to the use of compounds derived from betulin in the control of plant pests as efficient antifeedant agents. The invention is further directed to environmentally acceptable novel betulin derivatives comprising natural compounds and/or known compounds with low toxicity as substituents, such as to alcohol, phenol and/or carboxylic acid and/or ester and/or amide and/or ether derivatives of betulin, and moreover, to methods for the production thereof.

### DETAILED DESCRIPTION OF THE INVENTION

In the studies it was found that some environmentally acceptable compounds derived from betulin have wide range antifeedant activity on plant pests, particularly on the larvae of butterflies, such as cabbage moth, cabbage white and diamond-back moth, beetles such as cabbage beetle, and beetles of the genus Galerucella, and snails. Especially for snail control, potent synthetic pesticides are traditionally used, which may now at least partly be replaced with compounds according to the invention.

It is also possible to produce environmentally acceptable and efficient antifeedant agents from betulin and derivatives thereof for plant pests, said agents not only having desired activity but also superior solubility and/or miscibility and/or emulsifiability in media used in applications to control plant pests.

According to the invention, suitable compounds for antifeedant applications for plant pests include the following compounds derived from betulin having the general formula I shown below and salts thereof, where in formula I

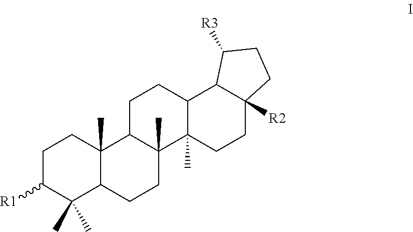

R1=—OH, —ORₐ where Rₐ represents C₁-C₁₂ aliphatic, unbranched or branched, saturated or unsaturated hydrocarbon residue; oxo (═O); —O(C═O)R_{b} where R_{b} represents C₁-C₂₂ aliphatic, unbranched or branched, saturated or unsaturated hydrocarbon residue; or a carboxymethyl, carboxymethylester or carboxymethylamide derivative;

R2=—CH₂OR_{c} where R_{c} represents H or —(C═O)R_{d} where R_{d} represents C₁-C₂₂ aliphatic, unbranched or branched, saturated or unsaturated hydrocarbon residue; a benzyl, phenyl, amino acid or anhydride derivative; or —(C═O)ORₑ where Rₑ represents H, C₁-C₁₂ aliphatic, unbranched or branched, saturated or unsaturated hydrocarbon residue; benzyl, phenyl or amino acid amide derivative; or a carboxymethyl, carboxymethylester or carboxymethylamide derivative; and

R3=isopropenyl, isopropyl, isopropylphenyl, isopropylhydroxyphenyl, or isopropylsuccinic acid derivative.

Preferable compounds are compounds IA-IL wherein the groups R1 to R3 represent the following structures:

IA:

R1=OH;

R2=CH₂O(C═O)R_{f} where R_{f}=C₁₁-C₂₂ linear or branched alkyl or alkenyl group;

and

R3=CH₂═CCH₃ (isopropenyl group)

IB:

R1=OH;

R2=CH₂O(C═O)(CHR_{g})CH₂COOY where R_{g}=C₄-C₂₂ linear or branched alkyl or alkenyl group, Y=H, Na, K, Ca, Mg, C₁-C₄-alkyl group, or NRₕ where Rₕ=H or C₁-C₄-alkyl group; and

R3=CH₂═CCH₃

IC:

R1=OH;

R2=CH₂ORᵢ where Rᵢ=an ester of ornithine, an ester of nicotine, an ester of N-acetylanthranilic acid or an ester of trimethylglycine (or betain ester); and

R3=CH₂═CCH₃

ID:

R1=OH;

R2=CH₂O(C═O)CHRⱼ(NHZ) where Rⱼ=H, C₁-C₄-alkyl, benzyl, 4-hydroxybenzyl, —CH₂CH₂CH₂CH₂NH₂, 4-imidazolylmethyl, 3-indolylmethyl or a CH₃SCH₂ group; and Z=H, Rₖ, (C═O)Rₖ or COORₖ where Rₖ=C₁-C₂₂ branched or unbranched alkyl or alkenyl group, or a phenyl, benzyl or 4-hydroxybenzyl group; and

R3=CH₂═CCH₃

I.E.:

R1=OH;

R2=CH₂ORₙ where Rₙ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid; and

R3=CH₂═CCH₃

IFa:

R1=O(C═O)Rₘ where Rₐ=C₁₁-C₂₂ linear or branched alkyl or alkenyl group or a C₃ -C₂₂ cyclic aliphatic, branched or unbranched, saturated or unsaturated hydrocarbon residue or a benzyl or phenyl group;

R2=CH₂O(C═O)Rₒ where Rₒ=C₁₁-C₂₂ linear, cyclic or branched alkyl or alkenyl group or a C₃-C₂₂ cyclic aliphatic, branched or unbranched, saturated or unsaturated hydrocarbon residue or a benzyl or phenyl group; and

R3=CH₂═CCH₃

IFb:

R1=O(C═O)(CHR_{c})CH₂COOY where R_{c}=C₄-C₂₂ linear or branched alkyl or alkenyl group, Y=H, Na, K, Ca, Mg, C₁-C₄ alkyl group or NRₕ where Rₕ=H or a C₁-C₄ alkyl group;

R2=CH₂O(C═O)(CHR_{d})CH₂COOY where R_{d}=C₄-C₂₂ linear or branched alkyl or alkenyl group, Y=H, Na, K, Ca, Mg, C₁-C₄ alkyl group or NRₖ where Rₖ=H or a C₁-C₄ alkyl group; and

R3=CH₂═CCH₃

IFc:

R1=ORᵣ where Rᵣ=an ester of ornithine, an ester of N-acetylanthranilic acid, or an ester of trimethylglycine or betaine ester;

R2=CH₂ORₚ where Rₚ=an ester of ornithine, an ester of N-acetylanthranilic acid or an ester of trimethylglycine or betaine ester ester; and

R3=CH₂═CCH₃

IFd:

R1=O(C═O)CHRₛ(NHZ) where Rₛ=CH₂CH₂CH₂CH₂NH₂, 4-imidazolylinethyl or 3-indolylmethyl group and Z=H, Rₖ, (C═O)Rₖ or COORₖ where Rₖ=C₁-C₂₂ branched or unbranched alkyl or alkenyl group or a phenyl, benzyl or 4-hydroxybenzyl group;

R2=CH₂O(C═O)CHRₓ(NHZ) where Rₓ=CH₂CH₂CH₂CH₂NH₂, 4-imidazolylmethyl or 3-indolylmethyl group and Z=H, R_{y}, (C═O)R_{y} or COOR_{y} where R_{y}=C₁-C₂₂ branched or unbranched alkyl or alkenyl group or a phenyl, benzyl or 4-hydroxybenzyl group; and

R3=CH₂═CCH₃

IFe:

R1=ORᵥ where Rᵥ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R2=CH₂ORᵤ where Rᵤ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid; and

R3=CH₂═CCH₃

IG:

R1=OH;

R2=(C═O)NHCHRₓCOOY where Y=H, Na, K, Ca, Mg, C₁-C₄ alkyl group or NR_{y} where R_{y}=H or a C₁-C₄ alkyl group and Rₓ=CH₂CH₂CH₂CH₂NH₂, 4-imidazolylmethyl, 3-indolylmethyl group; and

R3=CH₂═CCH₃

IH:

R1=OH;

R2=(C═O)R_{w} where R_{w}=an ester of verbenol, terpineol, thymol, carvacrol, menthol, cinnamic alcohol, curcumin, eugenol, borneol, or isoborneol; and

R3=CH₂═CCH₃

IIa:

R1=OR_{z} where R_{z}=CH₂CH₂CH₂CH₂NH₂, 4-imidazolylmethyl or 3-indolylmethyl group, or an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R2=(C═O)NHCHRₓCOOY where Y=H, Na, K, Ca, Mg, C₁-C₄ alkyl group or NR_{y} where R_{y}=H or a C₁-C₄ alkyl group and Rₓ=H, C₁-C₄-alkyl, benzyl, 4-hydroxybenzyl, —CH₂CH₂CH₂CH₂NH₂, 4-imidazolylmethyl, 3-indolylmethyl or a CH₃SCH₂ group; and

R3=CH₂═CCH₃

IIb:

R1=ORₐ where Rₐ=H, C₁-C₄ alkyl, benzyl, 4-hydroxybenzyl, CH₂CH₂CH₂CH₂NH₂ or a CH₃SCH₂ group, or an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R2=(C═O)R_{w} where R_{w}=an ester of verbenol, terpineol, thymol, carvacrol, menthol, cinnamic alcohol, curcumin, eugenol, borneol or isoborneol; and

R3=CH₂═CCH₃

IJa:

R1=oxo(=O) group;

R2=(C═O)NHCHRₓCOOY where Y=H, Na, K, Ca, Mg, C₁-C₄ alkyl group or NR_{y} where R_{y}=H or a C₁-C₄ alkyl group, and Rₓ=H, C₁-C₄-alkyl, benzyl, 4-hydroxybenzyl, CH₂CH₂CH₂CH₂NH₂, 4-imidazolylmethyl, 3-indolylmethyl or a CH₃SCH₂ group; and

R3=CH₂═CCH₃

IJb:

R1=oxo group;

R2=(C═O)R_{w} where R_{w}=an ester of verbenol, terpineol, thymol, carvacrol, menthol, cinnamic alcohol, curcumin, eugenol, borneol, or isoborneol; and

R3=CH₂═CCH₃

IK:

R1=OH or O—(C═O)R_{b} where R_{b}=C₁-C₂₂ alkyl or C₁-C₂₂ alkenyl group or a phenyl group;

R2=CH₂OH or CH₂O—(C═O)R_{f} where R_{f}=C₁-C₂₂ alkyl or C₁-C₂₂ alkenyl group or a phenyl group; and

R3=(CH₃)₂CR_{z} or CH₃CHCH₂R_{z} where R_{z}=C₆H₅₋ₙ(OH)ₙ or C₆H₅₋ₙ₋ₘ—(OH)ₙ(OCH₃)ₘ and n=0-5, m=0-5, n+m≦5

IL:

R1=OH or O—(C═O)R_{b} where R_{b}=C₁-C₂₂ alkyl or C₁-C₂₂ alkenyl group or a phenyl group;

R2=CH₂OH or CH₂O—(C═O)R_{f} where R_{f}=C₁-C₂₂ alkyl or C₁-C₂₂ alkenyl group or a phenyl group; and

R3=H₂C═CCH₂R_{q} or CH₃CCH₂R_{q} where R_{q}=succinic anhydride, succinic imide or CH(COOR_{q})CH₂COOR₂ where Rₒ=H, Na, K, Ca, Mg or a C₁-C₂₂ linear or branched alkyl or alkenyl group and R_{z}=H, Na, K, Ca, Mg or a C₁-C₂₂ linear or branched alkyl or alkenyl group

Substituents present in the compounds derived from betulin presented above are typically derived from naturally occuring substances or compounds known as exhiting low toxicity, or both. The present compounds derived from betulin are environmentally acceptable compounds having only weak potential negative effects in antifeedant applications for plant pests, said negative effects being also more predictable that those of synthetic compounds generally. Decomposition of compounds derived from betulin typically yields betulin or acid derivatives thereof, and further, constituents of substituents. Decomposition pathways of constituents, such as natural substances, present as structural moieties in the compounds and products thus generated are well known. Moreover, the toxicity of compounds derived from betulin is low as demonstrated by the cytotoxicity studies performed for instance in the examples below.

In studies it was shown that the compounds derived from betulin, presented above surprisingly have potent antifeedant activity for plant pests such as butterfly pests, beetles, e.g. cabbage beetle, or beetles of the genus Galerucella, and snails or slugs. Said compounds are particularly efficient against butterflies (Lepidoptera) selected from the group consisting of members belonging to the subfamily Hadeninae of the moth family (Noctuidae), such as cabbage moth (Mamestra brassicae), members of the family daybutterflies (Rhopalocera), such as cabbage white (Pieris brassicae), moths belonging to the suborder of little butterflies (Micro-lepidoptera), such as diamondback moth (Plutella xylostella), against beetles (Coleoptera) selected from the suborders Alticinae of leafbeetles (Chrysomelidae), such as rapeseed beetle (Phyllotreta spp.), strawberry and cloudberry beetles (Galerucella spp.), lily beetles (Lilioceris lilii) of the Criocerinae order and against gastropodes (Gastropoda), such as field snail and false field snail (Deroceras spp.)

Preferable antifeedant compounds for plant pests are selected from the group consisting of betulin 3,28-O-isostearylic acid diester, betulin 28-O-isostearylic acid ester, Betulin 3,28-O-oleinylic acid diester, betulin 28-O-oleinylic acid ester, betulin 3,28-O-octanylic acid diester, betulin 28-O-octanylic acid ester, 3,28-diacetoxy betulin, 28-acetoxy betulin, 3-oxo-28-acetoxy betulin, betulinic acid, 3-dehydroxide betulin, 3-dehydroxide-28-acetoxy betulin, betulonic acid, betulin, betulin 28-N-acetylanthralinic acid ester, behilin 28-nicotinic acid ester, betulin 3,28-C₁₈-alkylenesuccinic acid diester, betulin 28-C₁₈-alkylenesuccinic acid ester, betulin 28-carboxymethoxy menthol, betulin 28-carboxymethoxy thymol ester, betulin 28-chrysantemate, betulin 28-cinnamic acid ester, betulin L-aspartate amide, betulinic acid L-histidin amide, betulinic acid L-glutamin amide, betulinic acid L-lycin amide and betulonic acid 28-aspartate amide dimethylester.

Particularly preferable compounds are betulin, betulinic acid, betulonic acid, succinic acid derivatives of betulin such as betulin 28-C₁₈-alkylsuccinic acid ester and betulin 28-carboxymethoxy menthol.

New, environmentally acceptable compounds derived from betulin include the following betulin derivatives according to the general formula I shown below, and salts thereof, where in formula I

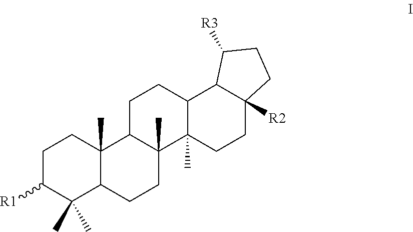

R1=—OH, —ORₐ where Rₐ represents C₃-C₂₂ cyclic, aliphatic, unbranched or branched, saturated or unsaturated hydrocarbon residue or a benzyl, phenyl or anhydride derivative; oxo (═O); —O(C═O)R_{b} where R_{b} represents C₃-C₂₂ cyclic, aliphatic, unbranched or branched, saturated or unsaturated hydrocarbon residue or a benzyl, phenyl or anhydride derivative; or a carboxymethyl, carboxymethylester or carboxymethylamide derivative;

R2=—CH₂OR_{c} where R_{c} represents H or C₃-C₂₂ cyclic, aliphatic, unbranched or branched, saturated or unsaturated hydrocarbon residue or a benzyl, phenyl or anhydride derivative, or R_{c} represents —(C═O)R_{d} where R_{d} represents C₃-C₂₂ cyclic, aliphatic, or heterocyclic, unbranched or branched, saturated or unsaturated hydrocarbon residue; a benzyl, phenyl, an anhydride derivative or an amino acid derivative with the proviso that R1 presents at the same time oxo (═O); or R2=-(C═O)ORₑ where Rₑ represents H, C₃-C₂₂ cyclic, aliphatic, unbranched or branched, saturated or unsaturated hydrocarbon residue; benzyl, phenyl or an anhydride derivative, or a carboxymethyl, carboxymethylester or carboxymethylamide derivative; and

R3=isopropenyl, isopropyl, isopropylphenyl, isopropythydroxyphenyl, or isopropylsuccinic acid derivative; and

with the proviso that the compound is not betulin, betulinic acid or betulonic acid.

Preferable new betulin derivatives according to formula I are compounds where R1 is selected from the following groups: OH; oxo group (═O); group O(C═)Rₘ; where Rₘ represents a C₃-C₂₂ cyclic, aliphatic, unbranched or branched, saturated or unsaturated hydrocarbon residue or a benzyl or phenyl group; group O(C═O)(CHR_{c})CH₂COOY where R_{c}=C₄-C₂₂ linear or branched alkyl or alkenyl group, Y=H, Na, K, Ca, Mg, C₁-C₄ alkyl group or NRₕ where Rₕ=H or a C₁-C₄ alkyl group; group ORᵣ where Rᵣ=an ester of ornithine, an ester of N-acetylanthranilic acid, or an ester of trimethylglycine; group O(C═O)CHRₛ(NHZ) where Rₛ=CH₂CH₂CH₂CH₂NH₂, 4-imidazolylmethyl or 3-indolylmethyl group and Z=H, Rₖ, (C═O)Rₖ or COORₖ where Rₖ=C₁-C₂₂ branched or unbranchcd alkyl or alkenyl group or a phenyl, benzyl or 4-hydroxybenzyl group; group ORᵥ where Rᵥ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid; group OR_{y}, where R_{y} represents C₃-C₂₂ cyclic, aliphatic, unbranched or branched, saturated or unsaturated hydrocarbon residue, or a benzyl, 4-hydroxybenzyl, CH₂CH₂CH₂CH₂NH₂, 4-imidazolylmethyl or 3 -indolylmethyl group; and

R2 is selected from the following groups: CH₂OH; group CH₂O(C═O)R_{f} where R_{f}=C₃-C₂₂ cyclic, aliphatic, branched or unbranched, saturated or unsaturated hydrocarbon residue; group CH₂O(C═O)(CHR_{g})CH₂COOY where R_{g}=C₄-C₂₂ linear or branched alkyl or alkenyl group and Y=H, Na, K, Ca, Mg, C₁-C₄ alkyl group or NRₕ where Rₕ=H or a C₁-C₄ alkyl group; group CH₂ORᵢ where Rᵢ=an ester of ornithine, an ester of N-acetylanthranilic acid or an ester of trimethylglycine; group CH₂O(C═O)CHRⱼ(NHZ) where Rⱼ=CH₂CH₂CH₂CH₂NH₂, 4-imidazolylmethyl or 3-indolylmethyl group and Z=H, Rₖ; (C═O)Rₖ or COORₖ where Rₖ=C₁-C₂₂ branched or unbranched alkyl or alkenyl group or a phenyl, benzyl or 4-hydroxybenzyl group; group CH₂ORₙ where Rₙ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid; group (C═O)NHCHRₓCOOY where Y=H, Na, K, Ca, Mg, C₁-C₄ alkyl group or NR_{y} where R_{y}=H or a C₁-C₄ alkyl group, and Rₓ=CH₂CH₂CH₂CH₂NH₂, 4-imidazolylmethyl or 3-indolylmethyl group; group (C═O)R_{w} where R_{w}=an ester of verbenol, terpineol, thymol, carvacrol, menthol, cinnamic alcohol, curcumin, eugenol, borneol, or isoborneol; group OR_{y}, where R_{y} represents C₃-C₂₂ cyclic, aliphatic, branched or unbranched, saturated or unsaturated hydrocarbon residue, benzyl, 4-hydroxybenzyl group, CH₂CH₂CH₂CH₂NH₂, 4-imidazolylmethyl or 3-indolylmethyl group; group (C═O)ORₚ where Rₚ=H or C₃-C₂₂ cyclic, aliphatic, branched or unbranched, saturated or unsaturated hydrocarbon residue; and

R3 is selected from the following groups: CH₂═CCH₃, group (CH₃)₂CR_{z} or CH₃CHCH₂R_{z} where R_{z}=C₆H₅₋ₙ(OH)ₙ or C₆H₅₋ₙ₋ₘ—(OH)ₙ(OCH₃)ₘ and n=0-5, m=0-5, n+m≦5; group H₂C═CCH₂R_{q} or CH₃CCH₂R_{q} where R_{q}=succinic anhydride, succinic imide or CH(COORₒ)CH₂COOR_{z} where Rₒ=H, Na, K, Ca, Mg or a C₁-C₂₂ linear or branched alkyl or alkenyl group and R_{z}=H, Na, K, Ca, Mg or a C₁-C₂₂ linear or branched alkyl or alkenyl group; and the above presented limitations concern also the preferable compounds.

Preferable compounds according to the invention are compounds according to types IA-IL, wherein the groups R1 to R3 represent following structures:

IA:

R1=OH;

R2=CH₂O(C═O)R_{f} where R_{f}=C₃-C₂₂ cyclic, aliphatic, branched or unbranched, saturated or unsaturated hydrocarbon residue or a benzyl or phenyl group; and

R3=CH₂═CCH₃ (isopropenyl group)

IB:

R1=OH;

R2=CH₂O(C═O)(CHR_{g})CH₂COOY where R_{g}=C₄-C₂₂ linear or branched alkyl or alkenyl group, Y=H, Na, K, Ca, Mg, C₁-C₄-alkyl group, or NRₕ where Rₕ=H or C₁-C₄-alkyl group; and

R3=CH₂═CCH₃

IC:

R1=OH;

R2=CH₂ORᵢ where Rᵢ=an ester of ornithine, an ester of N-acetylanthranilic acid or an ester of trimethylglycine (or betaine ester); and

R3=CH₂═CCH₃

ID:

R1=OH;

R2=CH₂O(C═O)CHRⱼ(NHZ) where Rⱼ=CH₂CH₂CH₂CH₂NH₂, 4-imidazolylmethyl or 3-indolylmethyl group; and Z=H, Rₖ, (C═O)Rₖ or COORₖ where Rₖ=C₁-C₂₂ branched or unbranched alkyl or alkenyl group, or a phenyl, benzyl or 4-hydroxybenzyl group; and

R3=CH₂═CCH₃

IE:

R1=OH;

R2=CH₂ORₙ where Rₐ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid; and

R3=CH₂═CCH₃

IFa:

R1=O(C═O)Rₘ where Rₐ=C₃-C₂₂ cyclic, aliphatic, branched or unbranched, saturated or unsaturated hydrocarbon residue or a benzyl or phenyl group;

R2=CH₂O(C═O)Rₒ where Rₒ=C₃-C₂₂ cyclic, aliphatic, branched or unbranched, saturated or unsaturated hydrocarbon residue or a benzyl or phenyl group; and

R3=CH₂═CCH₃

IFb:

R1=O(C═O)(CHR_{c})CH₂COOY where R_{c}=C₄-C₂₂ linear or branched alkyl or alkenyl group, Y=H, Na, K, Ca, Mg, C₁-C₄ alkyl group or NRₕ where Rₕ=H or a C₁-C₄ alkyl group;

R2=CH₂O(C═O)(CHR_{d})CH₂COOY where R_{d}=C₄-C₂₂ linear or branched alkyl or alkenyl group, Y=H, Na, K, Ca, Mg, C₁-C₄ alkyl group or NRₖ where Rₖ═H or a C₁-C₄ alkyl group; and

R3=CH₂═CCH₃

IFc:

R1=ORᵣ where Rᵣ=an ester of ornithine, an ester of N-acetylanthranilic acid, or an ester of trimethylglycine;

R2=CH₂ORₚ where Rₚ=an ester of ornithine, an ester of N-acetylanthranilic acid, or an ester of trimethylglycine; and

R3=CH₂═CCH₃

IFd:

R1=O(C═O)CHRₛ(NHZ) where Rₛ=—CH₂CH₂CH₂CH₂NH₂, 4-imidazolylmethyl or 3-indolylmethyl group, Z=H, Rₖ, (C═O)Rₖ or COORₖ where Rₖ=C₁-C₂₂ branched or unbranched alkyl or alkenyl group, or a phenyl, benzyl or 4-hydroxybenzyl group;

R2=CH₂O(C═O)CHRₓ(NHZ) where Rₓ=CH₂CH₂CH₂CH₂NH₂, 4-imidazolylmethyl or 3-indolylmethyl group, Z32 H, R_{y}, (C═O)R_{y} or COOR_{y} where R_{y}=C₁-C₂₂ branched or unbranched alkyl or alkenyl group, or a phenyl, benzyl or 4-hydroxybenzyl group; and

R3=CH₂═CCH₃

IFe:

R1=OR where Rᵥ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R2=CH₂ORᵤ where Rᵤ=an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R3=CH₂═CCH₃

IG:

R1=OH;

R2=(C═O)NHCHRₓCOOY where Y=H, Na, K, Ca, Mg, C₁-C₄ alkyl group or NR_{y} where R_{y}=H or a C₁-C₄ alkyl group, and Rₓ=CH₂CH₂CH₂CH₂NH₂, 4-imidazolylmethyl or 3-indolylmethyl group; and

R3=CH₂═CCH₃

IH;

R1=OH;

R2=(C═O)R_{w} where R_{w}=an ester of verbenol, terpineol, thymol, carvacrol, menthol, cinnamic alcohol, curcumin, eugenol, borneol, or isoborneol; and

R3=CH₂═CCH₃

IIa:

R1=OR_{z} where R_{z}=CH₂CH₂CH₂CH₂NH₂, 4-imidazolylmethyl or 3-indolylmethyl group, or an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R2=(C═O)NHCHRₓCOOY where Y=H, Na, K, Ca, Mg, C₁-C₄ alkyl group or NR_{y} where R_{y}=H or a C₁-C₄ alkyl group, and Rₓ=CH₂CH₂CH₂CH₂NH₂, 4-imidazolylmethyl or 3-indolylmethyl group; and

R3=CH₂═CCH₃

IIb:

R1=ORₐ where Rₐ=H, benzyl, 4-hydroxybenzyl, CH₂CH₂CH₂CH₂NH₂ group, 4-imidazolylmethyl or 3-indolylmethyl group; or an ester of carboxymethoxy substituted verbenol, terpineol, thymol, carvacrol, menthol, cinnamic alcohol, curcumin, eugenol, borneol, isoborneol, or an ester of chrysanthemic acid, cinnamic acid or retinolic acid;

R2=(C═O)R_{w} where R_{w}=an ester of verbenol, terpineol, thymol, carvacrol, menthol, cinnamic alcohol, curcumin, eugenol, borneol, or isoborneol; and

R3=CH₂═CCH₃

IJa:

R1=oxo(═O) group;

R2=(C═O)NHCHRₓCOOY where Y=H, Na, K, Ca, Mg, C₁-C₄ alkyl group or NR_{y} where R_{y}=H or a C₁-C₄ alkyl group, and Rₓ=CH₂CH₂CH₂CH₂NH₂, 4-imidazolylmethyl or 3 -indolylmethyl group; and

R3=CH₂═CCH₃

IJb:

R1=oxo group;

R2=(C═O)R_{w} where R_{w}=an ester of verbenol, terpineol, thymol, carvacrol, menthol, cinnamic alcohol, curcumin, eugenol, borneol, or isoborneol; and

R3=CH₂═CCH₃

IK:

R1=OH or O—(C═O)R_{b} where R_{b}=C₁-C₂₂ alkyl or C₁-C₂₂ alkenyl group or a phenyl group;

R2=CH₂OH or CH₂O—(C═O)R_{f} where R_{f}=C₁-C₂₂ alkyl or C₁-C₂₂ alkenyl group or a phenyl group; and

R3=(CH₃)₂CR_{z} or CH₃CHCH₂R_{z} where R_{z}=C₆H₅₋ₙ(OH)ₙ or C₆H₅₋ₙ₋ₘ—(OH)ₙ(OCH₃)ₘ and n=0-5, m=0-5, n+m≦5

IL:

R1=OH or O—(C═O)R_{b} where R_{b}=C₁-C₂₂ alkyl or C₁-C₂₂ alkenyl group or a phenyl group;

R2=CH₂OH or CH₂O—(C═O)R_{f} where R_{f}=C₁-C₂₂ alkyl or C₁-C₂₂ alkenyl group or a phenyl group; and

R3=H₂C═CCH₂R_{q} or CH₃CCH₂R_{q} where R_{q}=succinic anhydride, succinic imide or CH(COORₒCH₂COOR_{z} where Rₒ=H, Na, K, Ca, Mg or a C₁-C₂₂ linear or branched alkyl or alkenyl group and R_{z}=H, Na, K, Ca, Mg or a C₁-C₂₂ linear or branched alkyl or alkenyl group

Present novel compounds include monoesters and diesters containing the cyclic hydrocarbon part of betulin, ans aminoacid derivatives and terpenederivatives of betulin, betulonic acic and betulinic acid, such as anthranilic acid, chrysanthemic acid, ornithinic acid, cinnamic acid, retinolic acid, alpha-terpineol, verbenol, thymol, carvacrol, menthol, cinnamic alcohol, curcumin, eugenol, borneol, and isoborneol derivatives, and trimethyl glycine derivatives. Moreover, novel compounds of the invention include products and derivatives thereof obtained with subsequent reactions of 29-olefins of betulin such as with an alkylation reaction or an ene reaction, such as betulin succinate, phenol, and polyphenol derivatives.

Preferable novel compounds according to the invention include 28-C₁₈-alkylenesuccinic acid ester of betulin, 3,28-C₁₈-alkylenesuccinic acid diester of betulin, betulin 28-carboxymethoxy menthol, 28-carboxymethoxy thymol ester of betulin, betulin 28-chrysanthemate, 28-cinnamic acid ester of betulin, L-aspartate amide of betulinic acid, L-histidine amide of betulinic acid, L-glutamine amide of betulinic acid, L-lysine amide of betulinic acid, and 28-aspartate amide dimethyl ester of betulonic acid.

Particularly preferable novel compounds include 28-C₁₈-alkylenesuccinic acid ester of betulin, 3,28-C₁₈-alkylenesuccinic acid diester of betulin and betulin 28-carboxymethoxy menthol.

Compositions for the control of plant pests and particularly for antifeedant applications may be prepared from the compounds of the invention. At application concentration, said compositions contain between 0.01 and 50%, preferably between 0.1 and 10% of one or more compound(s) derined from betulin shown above, as the active antifeedant agent(s). Moreover, the composition may comprise excipients and additives known in the art. The composition is applied on the area to be treated or on cultivated plants to yield an amount of the active agent on the plant leaf of between 1 and 1000 μg/cm², preferably between 5 and 200 μg/cm² to provide the desired effect.

The present compounds derived from betulin may be emulsified, dissolved, or mixed in water, or in media used in applications to control plant pests using known mixing processes and additives. Suitable additives and media include for instance surfactants, emulsifying agents, dispersants, solvents, natural oils. The composition is optionally prepared while heating to provide a composition ready for use, or a concentrate to be diluted later with water or another diluent prior to use. Suitable media include acetone, alcohols like ethanol, plant oils and other substances acceptable for nature. Suitable plant oils include rapeseed, colza, tall, sunflower, palm, and olive oils. The present betulin derivatives may also, if necessary, be mixed in dry form to an inert carrier such as kaolin or talc, or applied as such on plants e.g. by dusting (spraying).

According to the invention, the compound derived from betulin is applied on plants in an amount ranging between 1 and 1000 μg/cm², preferably 5 and 200 μg/cm², optionally in combination with a carrier and/or a medium. Said betulin derivatives are applied on plants immediately after appearance of the pests, or in case a pest risk is foreseeable. Typically the application is performed for cabbage plants at the stage of cotyledons to control the cabbage beetles, or at the stage of small plants to control butterflies and moths, for strawberries in the spring right after the appearance of the Galerucella pests, and right after the lily plants have started their growth and during the summer in the case of Lilioceris Lilii. In order to prevent the damage caused by snails and gastropods the objects to be protected are sprayed typically immediately after there appear risk of snails or rains are forecasted.

Particularly betulin derivatives having long alkyl chains as substituents have a superior emulsifiability and/or solubility and/or miscibility in water or in media typically used in pesticidal applications such as in plant oils.

The solution according to the invention has several advantages. The compounds derived from betulin presented above are very useful for the control of plant pests since they are nontoxic to the plants and environment, and have no detrimental effects on defence mechanisms of the plants. The compounds are very biodegradable leaving no detrimental decomposition residues in nature. In addition, the compounds affect very specifically only the targeted organisms. According to the targeted microorganism and pesticidal application, the selectivity and decomposition rate of the agent may be controlled by substituents of betulin. If necessary, a compound decomposing more slowly, continuously releasing an active betulin component during decomposition may be prepared, thus achieving a uniform effect for a longer period of time.

Betulin derivatives of the invention described above may be produced by methods I-IX presented below.

Method I

Betulin esters of the type IB or IFb described above may be produced by reacting 1 mol of betulin with 0.8-1.5 moles, preferably 1-1.2 moles of a C₄-C₂₂ alkyl or alkenyl derivative of maleic anhydride in the presence of imidazol (1-7 moles, preferably 3-5 moles), and a solvent at 0 to 100° C., preferably at 20 to 70° C., for 5 to 100 hours, preferably 10 to 50 h. C₁₈ alkenyl succinic anhydride (ASA) is preferably used. N-methylpyrrolidone (NMP), N,N-dimethylformamide (DMF), dimethylsulfoxide (DMSO), 1,4-dioxane, diethyl ether, tetrahydrofuran (THF), acetone, ethyl acetate, hydrocarbons and/or chlorinated hydrocarbons or mixtures thereof, preferably NMP, may serve as the solvent. After completion of the reaction, the reaction mixture is allowed to cool to room temperature, followed by separation of the product for instance by pouring the mixture into water, decanting, dissolving in a solvent, and then if necessary, washing the product with a diluted hydrochloric acid solution and water. The solvent is removed e.g. by evaporation to dryness, thus yielding desired betulin ester as the crude product that may be purified by crystallization, chromatography or preferably by extraction using diethyl ether, tetrahydrofuran, 1,4-dioxane, 1,2-dimethoxy ethane, ethyl acetate, hydrocarbons and/or chlorinated hydrocarbons or mixtures thereof as the solvent. Esters corresponding to the structure IFb are obtained as the main product in case an excess of anhydride (1.6 to 5 moles, preferably 2 to 2.5 moles) is used, while the use of 1 to 1.2 moles of the anhydride yields esters corresponding to the structure IB.

Method II

Betulin esters having structures of types IA, IC, ID, IE, IFa, Ifc, IFd, and IFe described above may be produced from betulin (1 mol) and carboxylic acids (0.8 to 1.5 moles, preferably 1 to 1.2 moles) in the presence of N,N-dimethylamino pyridine (DMAP) (0.01 to 1 mol) and dicyclohexyl carbodiimide (DCC) (0.8 to 1.5 moles, preferably 1 to 1.2 moles), or N-(3-dimethylaminopropyl)-N′-ethylcarbodiimide hydrochloride (EDC) (0.8 to 1.5 moles, preferably 1 to 1.2 moles) and a solvent, by agitating at 0 to 60° C., preferably at 20 to 40° C. for 2 to 50 hours, preferably for 5 to 25 hours. The carboxylic acid is selected for different compound types as follows: IA: HO(C═O)Rᵢ where Rᵢ=C₁₁-C₂₂ linear or branched alkyl or alkenyl group; IC: ornithine, nicotine, and N-acetylanthranilic acid or trimethyl glycine; ID: HO(C═O)CRₓ(NHR_{y}); Rₓ=alkyl, heteroalkyl, or arylalkyl group; R_{y}=H or acyl group; and IE: a carboxymethoxy derivative of verbenol, terpineol, thymol, carvacrol, menthol, cinnamic alcohol, curcumin, eugenol, borneol, isoborneol; or chrysanthemic acid, cinnamic acid, or retinolic acid. NMP, DMF, DMSO, 1,4-dioxane, diethyl ether, tetrahydrofuran, 1,2-dimethoxy ethane, acetone, ethyl acetate, hydrocarbons and/or chlorinated hydrocarbons, or mixtures thereof; preferably dichloromethane, may serve as the solvent. After completion of the reaction, the reaction mixture is poured into water, organic layer is separated, followed by removing the solvent for instance by evaporation to dryness, thus yielding betulin ester as the crude product that may be purified if necessary by crystallization, chromatography, or extraction, preferably by extraction. Use of 0.8 to 1.5 moles of the carboxylic acid reagent results in compounds having the structures IA, IC, ID, IE or IFd while use of an excess of the carboxylic acid reagent (1.6 to 3 moles, preferably 2 to 2.5 moles) with dicyclohexyl carbodiimide (DCC) (1.6 to 3 moles, preferably 2 to 2.5 moles), or with N-(3-dimethylaminopropyl)-N′-ethylcarbodiimide hydrochloride (EDC) (1.6 to 3 moles, preferably 2 to 2.5 moles) yields compounds corresponding to structures IFa, IFc, IFd, or IFe. For the production of the compounds of the IE or IFe type, an acetic acid derivative of the alcohol used as starting material is first generated according to method V.

Method III

Betulin esters having structures of types IA, IC, IE, IFa, IFc, and IFd described above may be produced from betulin (1 mol) with carboxylic acids (0.8 to 1.5 moles, preferably 1 to 1.2 moles) in the presence of a tetraisopropyl ortho titanate, tetrabutyl ortho titanate, p-toluenesulfonic acid monohydrate, or pyridine-p-toluenesulfonate catalyst (0.01 to 1 mol), or sulphuric acid or hydrochloric acid (1 to 6%, preferably 2 to 4%) and a solvent, by agitating at 80 to 160° C., preferably at 100 to 140° C. for 2 to 50 hours, preferably for 4 to 25 hours. The carboxylic acid is selected for different compound types as follows: IA: HO(C═O)Rᵢ where Rᵢ=C₁₁-C₂₂ linear or branched alkyl or alkenyl group; IC: ornithine, nicotine, N-acetylanthranilic acid or trimethyl glycine; IE: a carboxymethoxy derivative of verbenol, terpineol, thymol, carvacrol, menthol, cinnamic alcohol, curcumin, eugenol, borneol, isoborneol; or chrysanthemic acid, cinnamic acid, or retinolic acid. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably toluene or xylene, may serve as the solvent. Water generated in the reaction is separated using a water separator tube, or vacuum. After completion of the reaction, the reaction mixture is poured into water, organic layer is separated, washed if necessary with a basic aqueous solution, preferably with an aqueous NaHCO₃ or Na₂CO₃ solution, followed by removing the solvent for instance by evaporation to dryness, thus yielding betulin ester as the crude product that may be purified if necessary by crystallization, chromatography, or extraction, preferably by extraction. Use of 0.8 to 1.5 moles of the carboxylic acid reagent results in compounds having the structures IA, IC, or IE while use of an excess of the carboxylic acid reagent (1.6 to 3 moles, preferably 2 to 2.5 moles) yields compounds corresponding to structures IFa, IFc or IFe. For the production of the compounds of the IE or IFe type, an acetic acid derivative of the alcohol used as starting material is first generated according to method V.

Method IV

Esters having structures of types IA, IC, ID, IE, IFa, Ifc, IFd, and IFe described above may be produced from betulin (1 mol) and carboxylic acids (0.8 to 1.5 moles, preferably 1 to 1.2 moles), first allowed to react with oxalyl chloride or thionyl chloride (1 to 10 moles, preferably 1 to 4 moles) without or in the presence of a solvent, by agitating at 0 to 80° C., preferably at 20 to 50° C. for 2 to 50 hours, preferably for 5 to 25 hours. The carboxylic acid is selected for different compound types as follows: IA: HO(C═O)Rᵢ where Rᵢ═C₁₁-C₂₂ linear or branched alkyl or alkenyl group; IC: ornithine, nicotine, N-acetylanthranilic acid or trimethyl glycine; HO(C═O)CRₓ(NHR_{y}); Rₓ=alkyl, heteroalkyl, or arylalkyl group; R_{y}=H or acyl group; and IE: a carboxymethoxy derivative of verbenol, terpineol, thymol, carvacrol, menthol, cinnamic alcohol, curcumin, eugenol, borneol, isoborneol; or chrysanthemic acid, cinnamic acid, or retinolic acid. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the solvent is removed for instance by evaporation to dryness, if necessary, followed by purification of the desired acid chloride by crystallization, chromatography, or extraction, preferably by extraction. The acid chloride (0.8 to 1.5 moles, perferably 1 to 1.2 moles) thus obtained is reacted with betulin (1 mol), base (0.5 to 10 moles, preferably 1 to 5 moles) such as triethyl amine, tripropyl amine, diisopropylethyl amine, preferably triethyl amine in the presence of a solvent, or in the presence of the DMAP catalyst (0.001 to 1 mol), pyridine and solvent, or with a base (0.5 to 10 moles, preferably 1 to 5 moles) such as triethyl amine, tripropyl amine, diisopropylethyl amine, preferably triethyl amine, and pyridine by agitating at 0 to 80° C., preferably at 20 to 50° C. for 2 to 50 hours, preferably for 5 to 25 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, betulin amide or betulin ester product is purified by crystallization, chromatography, or extraction, preferably by extraction, if necessary. Use of 0.8 to 1.5 moles of the acid chloride reagent results in compounds with the structures IA, IC, ID, or IE while use of an excess of the acid chloride reagent (1.6 to 3 moles, preferably 2 to 2.5 moles) yields compounds corresponding to structures IFa, IFc, IFd, or IFe. For the production of the compounds of IE or IFe type, an acetic acid derivative of the alcohol used as starting material is first generated according to method V.

Method V

For the production of betulin derivatives having structures of the IE and IFe type according to the methods II, III or IV, and betulin derivatives having structures of the IIa and IIb type according to the method IV, an acetic acid derivative of the alcohol is first generated as follows. Acetic acid derivative is produced by mixing an alcohol (1 mol) and chloroacetic acid (0.8 to 1.5 moles, preferably 1 to 1.2 moles) in water for 1 to 7 hours, preferably for 3 to 5 hours, at 100 to 150° C., preferably at 120-130° C., in the presence of lithium, potassium, sodium, or hydrides or hydroxides thereof (1.5 to 3 moles, preferably 1.8 to 2.2 moles), preferably sodium (Na), sodium hydride (NaH), or sodium hydroxide (NaOH). The alcohol is selected from the group consisting of verbenol, terpineol, thymol, carvacrol, menthol, cinnamic alcohol, curcumin, eugenol, borneol, and isoborneol. The mixture is allowed to cool to room temperature, made acidic with concentrated hydrochloric acid, and extracter with a solvent. Hydrocarbons and/or chlorinated hydrocarbons, diethyl ether, tetrahydrofuran, 1,4-dioxane, 1,2-dimethoxy ethane, ethyl acetate, or mixtures thereof, preferably diethyl ether, may serve as the solvent. If necessary, the organic phase is washed with a basic aqueous solution, preferably with an aqueous NaHCO₃ or Na₂CO₃ solution. The solvent is removed for instance by evaporation to dryness, thus yielding a carboxymethoxy intermediate that may be purified if necessary by crystallization, chromatography, or extraction, preferably by extraction.

Method VI

Derivatives of types IG, IH, II, and IJ described above may be produced from betulonic acid (1 mol) and natural alcohols (0.8 to 1.5 moles, preferably 1 to 1.2 moles), or amino acids (0.8 to 1.5 moles, preferably 1 to 1.2 moles), in the presence of a solvent and DMAP (0.001 to 1 moles) and DCC (0.8 to 1.5 moles, preferably 1 to 1.2 moles), or EDC (0.8 to 1.5 moles, preferably 1 to 1.2 moles), by agitating at 0 to 60° C., preferably at 20-50° C. for 2 to 50 hours, preferably for 5 to 25 hours. For the different compound types, the alcohol is selected as follows: IH: verbenol, terpineol, thymol, carvacrol, menthol, cinnamic alcohol, curcumin, eugenol, borneol, or isoborneol. For the different compound types, the amino acid is selected as follows: IG: HO(C═O)R_{f} where R_{f}═NHCHRₓCOOY where Y=H, Na, K, Ca, Mg, C₁-C₄-alkyl group or NRₓ where Rₓ=H, C₁-C₄-alkyl, benzyl, 4-hydroxybenzyl, —CH₂CH₂CH₂CH₂NH₂, 4-imidazolyl methyl, 3-indolyl methyl, or CH₃SCH₂ group; preferably dimethyl ester hydrochloride of aspartic acid, methyl ester hydrochloride of L-histidine, dimethyl ester hydrochloride of L-glutaminic acid, or methyl ester dihydrochloride of L-lysine. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably to dichloromethane, may serve as the solvent. After completion of the reaction, the desired betulonic acid amide or ester product (of the type IJa or IJb) may be purified by crystallization, chromatography, or extraction, preferably by extraction, if necessary. The betulonic acid amide or ester thus obtained may be reduced to the corresponding betulinic acid amide or ester product (of the type IG or IH) if desired using sodium borohydride according to U.S. Pat. No. 6,280,778. After completion of the reaction, said betulinic acid amide or ester may be purified by crystallization, chromatography, or extraction, preferably by extraction, if necessary. Betulin derivatives of the IIa and IIb type are obtained by reacting the betulinic acid amide or ester thus obtained as described in the methods II, III or IV.

Method VII

Compounds having structures of the types IC, IH, II, and IJ described above may be produced from betulonic acid (1 mol) by reacting with oxalyl chloride or thionyl chloride (1 to 10 moles, preferably 1 to 4 moles) without, or in the presence of a solvent by agitation at 0 to 80° C., preferably 20 to 50° C., for 2 to 50 hours, preferably for 5 to 25 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the desired acid chloride may be purified by crystallization, chromatography, or extraction, preferably by extraction, if necessary. Betulonic acid chloride thus obtained from the reaction (1 mol) is reacted with an amino acid (0.8 to 1.5 moles, preferably 1 to 1.2 moles), or an alcohol (0.8 to 1.5 moles, preferably 1 to 1.2 moles), with a base such as triethyl amine, tripropyl amide diisopropyl ethyl amine, pyridine, preferably triethyl amine in the presence of a solvent, or in the presence of the DMAP catalyst (0.001 to 1 mol), pyridine and solvent, or with a base (0.5 to 10 moles, preferably 1 to 5 moles) such as triethyl amine, tripropyl amine, diisopropylethyl amine, preferably triethyl amine, and pyridine by agitating at 0 to 80° C., preferably at 20 to 50° C. for 2 to 50 hours, preferably for 5 to 25 hours. For the different compound types, the amino acid is selected as follows: IG: HO(C═O)R_{f} where R_{f}=NHCHRₓCOOY where Y═H, Na, K, Ca, Mg, C₁-C₄-alkyl group or NRₓ where Rₓ=H, C₁-C₄-alkyl, benzyl, 4-hydroxybenzyl, —CH₂CH₂CH₂CH₂NH₂, 4-imidazolyl methyl, 3-indolyl methyl, or CH₃SCH₂ group; preferably dimethyl ester hydrochloride of aspartic acid, methyl ester hydrochloride of L-histidine, dimethyl ester hydrochloride of L-glutaminic acid, and methyl ester dihydrochloride of L-lysine. For the different compound types, the alcohol is selected as follows: IH: verbenol, terpineol, thymol, carvacrol, menthol, cinnamic alcohol, curcumin, eugenol, borneol, isoborneol, or eugenol. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably dichloromethane, may serve as the solvent. After completion of the reaction, the reaction mixture is washed with diluten hydrochloric acid solution and water. The solvent is evaporated to dryness, and the reaction product (of the type IJa or IJb) is purified by crystallization, chromatography, or extraction, preferably by extraction, if necessary. The betulonic acid amide or ester product thus obtained may be reduced to the corresponding betulinic acid amide or ester product (of the type IG or IH) using sodium borohydride according to U.S. Pat. No. 6,280,778. After completion of the reaction, the desired betulinic acid amide or ester is purified by crystallization, chromatography, or extraction, preferably by extraction, if necessary. Betulin derivatives of the II type are obtained by reacting the betulinic acid amide or ester thus obtained as described in the methods II, III or IV.

Method VIII

Compounds having structures of the type IK described above may be produced from betulin (1 mol) and aromatic compounds selected to have R_{z}=C₆H₅₋ₙ(OH)ₙ or C₆H₅₋ₙ₋ₘ(OH)ₙ(OCH₃)ₘ and n=0-5, m=0-5, n+m≦5 (4 to 20 moles) as the phenol residue in the IK group, in the presence of a polymeric acid catalyst, preferably a sulfonic acid derivative of polystyrene (0.1 to 1.5 g, preferably 0.5 to 1 g, 16 to 50 mesh) and a solvent. The reaction mixture is agitated in an inert atmosphere at 20 to 120° C., preferably at 75 to 110° C. for 1 to 5 hours, preferably for 2 to 4 hours. Water generated in the reaction is suitably separated using water separating tube or vacuum. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof, preferably hydrocarbons and/or chlorinated hydrocarbons or ether may serve as the solvent. After completion of the reaction, the mixture is allowed to cool to room temperature, filtered, the filtrate is washed with water, dried, and the solvent is separated. The betulin derivative thus obtained is purified by crystallization, chromatography, or extraction, preferably by extraction, if necessary.

Method IX

Compounds having structures of the type IL described above may be produced from compounds having structures of the type IA or IFa prepared as described in the methods II, III, or IV, and maleic anhydride (0.8 to 10 moles, preferably 1 to 5 moles), in the presence of hydrochinone (0.05 to 0.5 moles, preferably 0.08 to 0.3 moles), and a solvent, or in a melt by heating the reaction mixture at 150 to 220° C., preferably at 160 to 180° C. for 1 to 5 hours, preferably for 2 to 4 hours. Hydrocarbons and/or chlorinated hydrocarbons, NMP, DMF, DMSO, 1,4-dioxane, diethyl ether, tetrahydrofuran, 1,2-dimethoxy ethane, acetone, ethyl acetate, or mixtures thereof may serve as the solvent, preferably as a melt. After completion of the reaction, the desired product is purified by crystallization, chromatography, or extraction, preferably by extraction, if necessary. The obtained maleic anhydride derivative of betulin may be further converted into an imide or ester compound having the structure of the type IL with known methods.

The invention is now illustrated by the following examples without wishing to limit the scope thereof.

### EXAMPLES

### Example 1

### Preparation of 28-C₁₈ alkylene Succinic Ester of Betulin

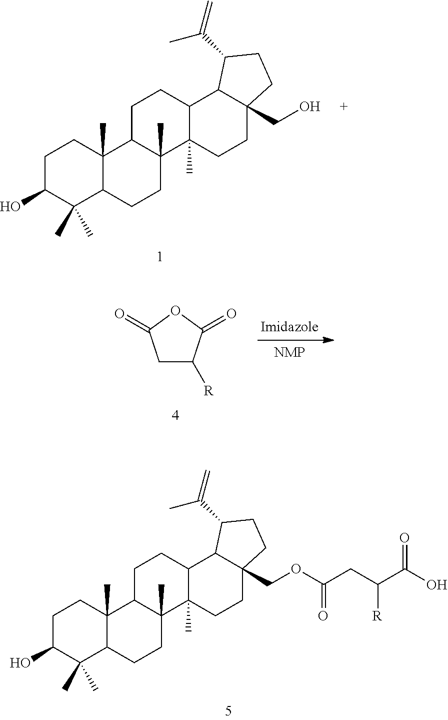

Imidazole (38.8 mmol) and C₁₈ alkylene succinic anhydride (ASA) 4 (11.6 mmol) were agitated in NMP (25 ml). Betulin 1 (9.7 mmol) was added, followed by further agitation at room temperature for 3 days. The organic phase was poured into water, decanted, dissolved in dichloromethane, and washed. The solvent was evaporated, thus yielding 28-C₁₈ alkylene succinic ester of betulin 5, yield 73%.

### Example 2

### Preparation of the 3,28-C₁₈ alkylene Succinic Diester of Betulin

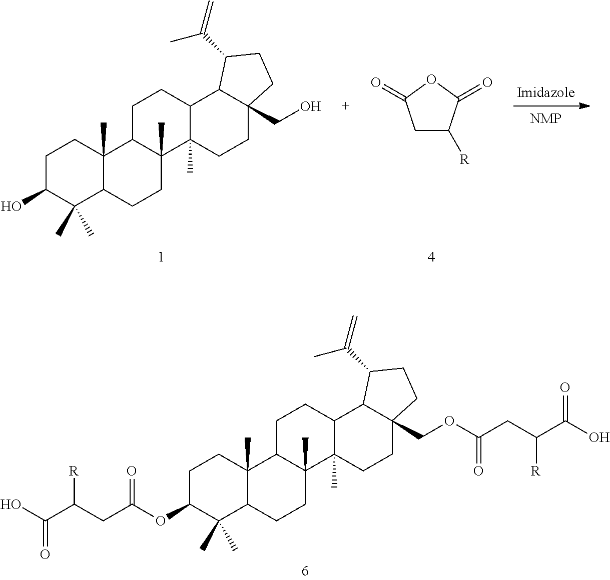

Imidazole (54.2 mmol) and C₁₈ alkylene succinic anhydride (ASA) 4 (32.5 mmol) were agitated in NMP (30 ml). Betulin 1 (13.5 mmol) was added, followed by further agitation of the mixture at RT for 3 days. The organic phase was poured into water, decanted, dissolved in dichloromethane, and washed. The solvent was evaporated yielding 3,28-C₁₈ alkylene succinic diester of betulin 6 with a yield of 40%.

### Example 3

### Preparation of the 28-carboxymethoxy Mentholester of Betulin

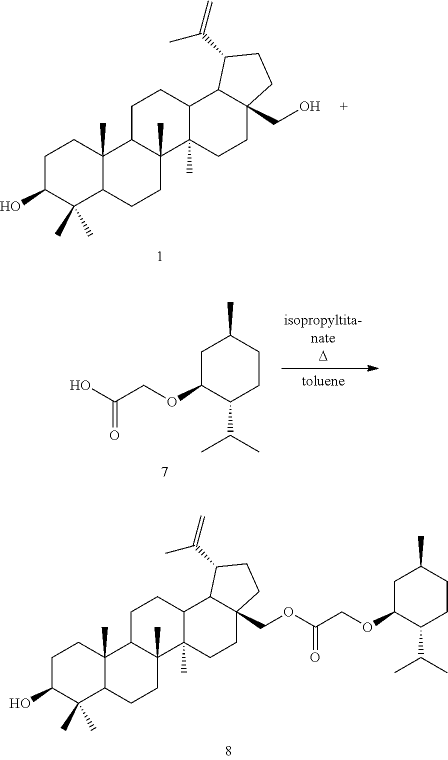

Betulin 1 (11.7 mmol) and menthoxyacetic acid 7 (11.7 mmol) were weighed in a flask, followed by the addition of toluene (120 ml). The mixture was heated to 120° C., and added with isopropyl titanate (1.4 mmol). The reaction mixture was refluxed for 3 h until water was separated by water separation tube. The mixture was cooled to room temperature and the precipitate formed was filtered. The organic phase was washed and the solvent was evaporated, yielding 28-carboxymethoxy mentholester of betulin 8 with a yield of 60%.

### Example 4

### Preparation of the 28-carboxymethoxy Carvacrolester of Betulin

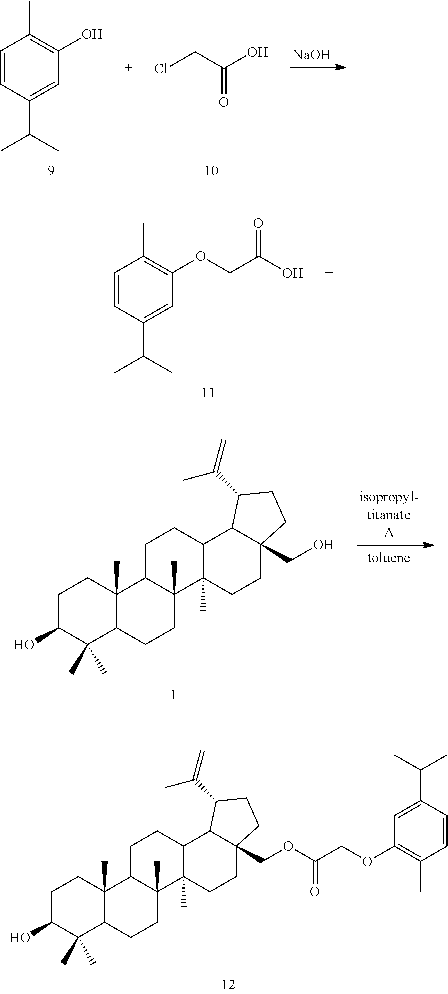

NaOH beads (66.6 mmol), dissolved in water, were added to a mixture of carvacrol 9 (33.3 mmol), chloroacetic acid 10 (33.3 mmol) and water (50 ml). The mixture was refluxed at 120° C. for 3 h. The mixture was cooled to room temperature and acidified with hydrochloric acid. The crude product was extracted with diethyl ether and washed with water. The solvent was evaporated, thus giving carvacrol oxyacetic acid 11, yield 83%. The crude product was purified by dissolving in diethyl ether, followed by extraction with water and NaHCO₃ solution. Aqueous phases were pooled, acidified with hydrochloric acid and extracted with diethyl ether. The ether phase was dried followed by evaporation of the solvent to dryness, thus giving carvacrol acetic acid 11, yield 45%. Betulin 1 (7.2 mmol) and carvacrol oxyacetic acid 11 (7.2 mmol) were weighed into a flask and toluene (80 ml) was added. The bath was heated to 160° C. and isopropyl titanate (1.4 mmol) was added. The reaction mixture was refluxed for 6 h until all water was separated by water separation tube. The mixture was cooled to RT and the precipitate formed was filtered. The organic phase was washed with NaHCO₃ solution and the solvent was evaporated. The crude product was recrystallized from boiling solution of cyclohexane and toluene. The solvent was evaporated to dryness, thus isolating 28-carboxymethoxy carvacrolester of betulin 12 yield 55%.

### Example 5

### Preparation of the 28-cinnamon Alcohol Acetic Acid Ester of Betulin

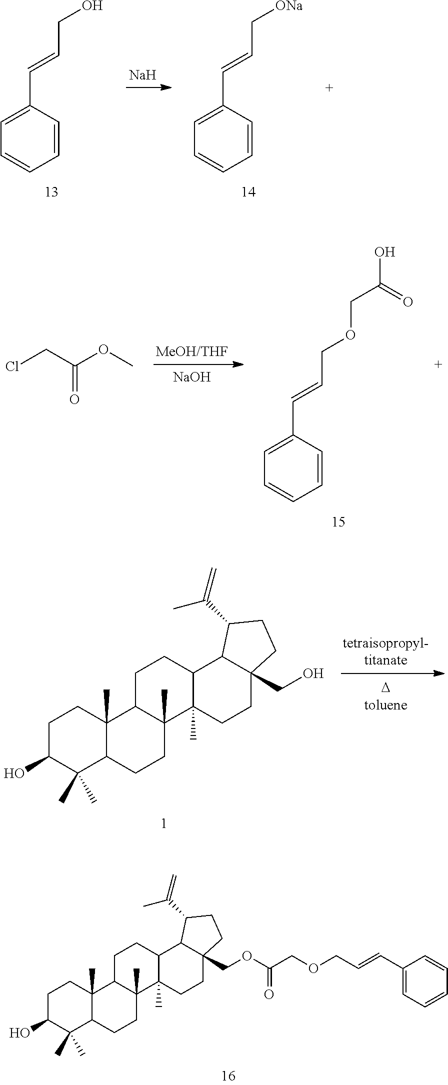

A mixture of sodium hydride (8.2 mmol) and tetrahydrofuran was added with cinnamon alcohon 13 (7.5 mmol), and agitation was continued for 1 hour. Methyl-chloroacetate (7.5 mmol) was added to the reaction flask, and agitation was continued for 24 hours. After completion of the reaction, the reaction mixture was diluted with diethyl ether, and then the organic phase was washed with water and dried. The solvent was evaporated to dryness, and the precipitate was dissolved in a solution of methanol and tetrahydrofuran. Sodium hydroxide solution (10.9 mmol) was added, and the reaction mixture was refluxed for 4 hours. The solvent was evaporated. Water was added to the flask, acidified with hydrochloric acid, and extracted with diethyl ether. The organic phase was washed with water, and the solvent was evaporated, thus giving cinnamic acid 15, yield 23%. Betulin 1 (0.9 mmol) and cinnamic acid 15 (0.9 mmol) were weighed into a flask, and toluene (40 ml) was added as the solvent. The bath was heated to 160° C., and then isopropyl titanate (0.2 mmol) was added to the reaction mixture. The reaction mixture was refluxed for 4.5 h until all water was separated by water separation tube. The mixture was cooled to room temperature and the precipitate formed was filtered. The organic phase was washed with NaHCO₃ solution and the solvent was evaporated. The crude product was recrystallized from boiling solution of cyclohexane and toluene. After the mixture was cooled, the crystallized precipitate was filtered. The solvent was evaporated to dryness, thus giving 28-cinnamon alcohol acetic acid ester of betulin 16 with a yield of 14%.

### Example 6

### Preparation of 28-eugenolester of Betulonic Acid

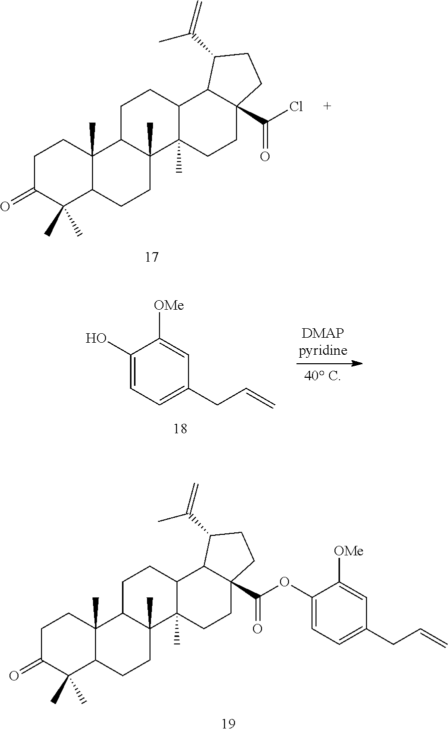

A mixture of betulonic acid chloride 17 (1.4 mmol) (prepared as described in example 12), eugenol 18 (1.1 mmol), DMAP (1.1 mmol), and pyridine was heated for 48 hours at 40° C. After completion of the reaction, the reaction mixture was diluted with toluene, washed with diluted hydrochloric acid solution, and water and then dried over sodium sulfate. The solvent was evaporated, thus giving 28-eugenol ester of betulonic acid 19 with a yield of 81%.

### Example 7

### Preparation of 28-carboxymethoxythymol Ester of Betulin

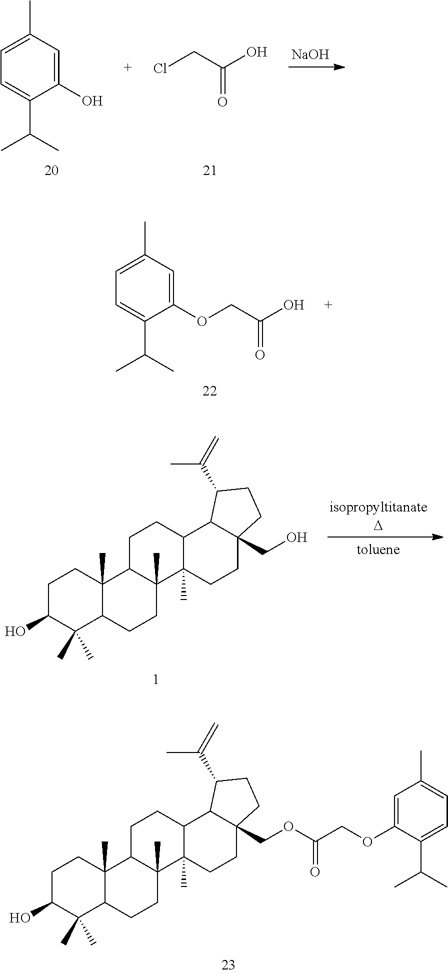

NaOH beads (66.6 mmol), dissolved in water, were added to a mixture of thymol 20 (33.3 mmol), chloroacetic acid 21 (33.3 mmol) and water. The mixture was refluxed at 120° C. for 3 h. The mixture was cooled to room temperature, acidified, extracted with diethyl ether and washed. The solvent was evaporated thus giving precipitated thymolacetic acid 22 with a yield of 29%. Betulin 1 (7.2 mmol), thymolacetic acid 22 (7.2 mmol), and toluene (80 ml) were heated to 160° C., followed by the addition of isopropyl titanate (1.4 mmol) to the reaction mixture. The reaction mixture was refluxed for 4.5 h until all water was separated by water separation tube. The mixture was cooled to room temperature and the precipitate formed was filtered. The organic phase was washed and the solvent was evaporated. The crude product was recrystallized from boiling solution of cyclohexane and toluene (3.5:1), thus giving 28-carboxymethoxythymol ester of betulin 23 with a yield of 61%.

### Example 8

### Preparation of 28-chrysanthemate of Betulin

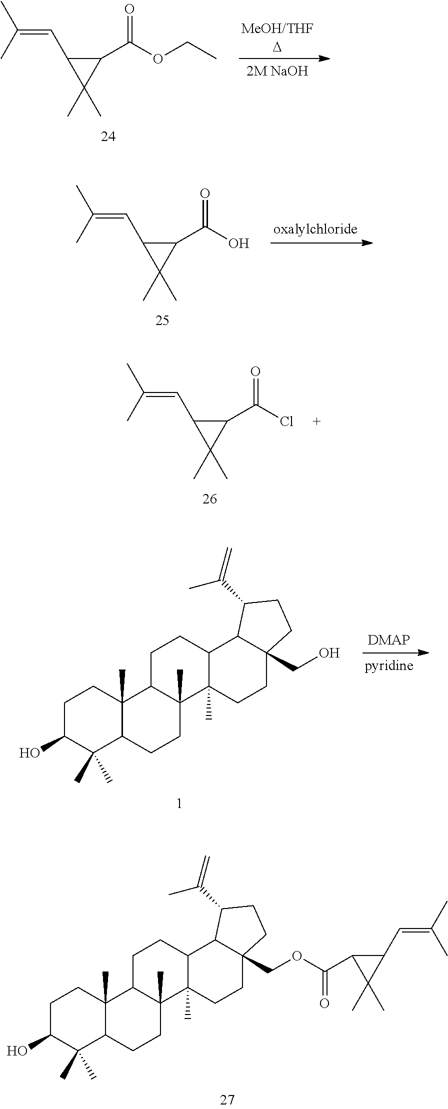

Ethyl chrysanthemate 24 (23.3 mmol) was mixed to a THF/MeOH solution (1:2) under an inert atmosphere. 2 M NaOH solution (93 ml) was slowly added to the mixture, and then, the reaction mixture was heated at 80° C. for 4 hours until no starting material was present as determined by TLC (hexane:ethyl acetate 6:1, 5% by volume of acetic acid). The solvent was evaporated, the crude product obtained was dissolved in water (400 ml) and extracted with diethyl ether. The aqueous phase was acidified with hydrochloric acid, and diluted with diethyl ether. The ether phase was washed and the solvent was evaporated in vacuum, thus giving chrysanthemic acid 25 with a yield of 90%.

Chrysanthemic acid 25 (5.9 mmol) in anhydrous dichloromethane (30 ml) was added with oxalyl chloride (11.8 mmol) at room temperature under inert atmosphere. After six hours, the solvent was evaporated, and then the evaporation residue was taken up in dry dichloromethane, which was again evaporated. The procedure was repeated three times, thus giving chrysanthemic acid chloride 26 with a yield of 81%.

Betulin 1 (0.9 mmol), chrysanthemic acid chloride 26 (1.1 mmol) and DMAP (0.9 mmol) were agitated in pyridine at 40° C. under inert atmosphere for 48 hours. EtOAc (100 ml) was added, organic phase was washed with water, the solvent was evaporated, and the residue was recrystallized in cyclohexane. 28-chrysanthemate of betulin 27 was obtained with a yield of 63%.

### Example 9

### Preparation of 28-cinnamic Acid Ester of Betulin

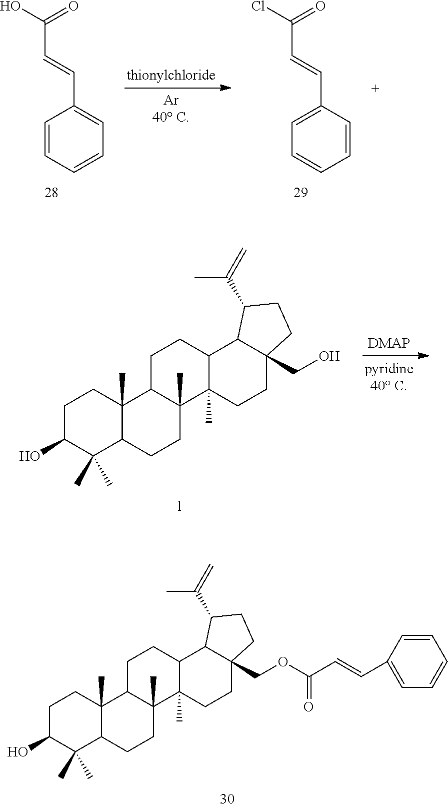

Cinnamic acid 28 (18.06 mmol) and thionyl chloride (180.6 mmol) were mixed under argon atmosphere at 40° C. for 24 hours. Solvent was evaporated under vacuum, followed by dissolving the evaporation residue twice in dichloromethane and evaporation, thus giving cinnamic acid chloride 29 with a yield of 99%.

Betulin 1 (5.4 mmol) and cinnamic acid chloride 29 (5.6 mmol) were agitated in dry pyridine (80 ml) in the presence of DMAP (5.6 mmol) under inert argon at mosphere at 40° C. for 24 hours. Toluene (100 ml) was added, and the organic phase was washed. Solvent was evaporated, followed by purification of the crude product by recrystallization in a cyclohexane/toluene mixture (5:1) and extraction. 28-cinnamic acid ester of betulin 30 was obtained with a yield of 67%.

### Example 10

### Preparation of Fatty Acid Esters of Betulin

Betulin 1 (5 mmol) and fatty acid (5 mmol) were weighed in a flask equipped with a water separation tube. Toluene and a catalytic amount of isopropyl titanate or p-toluenesulphonic acid monohydrate were added, followed by refluxing the reaction mixture in an oil bath for about 5 hours. The reaction mixture was allowed to cool to room temperature, the organic layer was washed with sodium hydrogen carbonate solution, separated, dried over sodium sulphate, and then the solvent was evaporated to dryness. The crude product obtained, betulin monoester, was purified by chromatography, if necessary. In case 2 equivalents of the fatty acid and 1 equivalent of betulin were used, also betulin diesters were obtained as the product as shown in table 1. Table 1 shows yields of the esterification reactions of betulin with fatty acids, and degrees of esterification.

TABLE 1

| | | | Total | Degree of C₃ | |
|---|---|---|---|---|---|
| Fatty | | Reflux | yield | esterification | Degree of C₂₈ |
| acid | Catalyst | time (h) | (%) | (%) | esterification (%) |
| Isostearic | Isopropyl | 3 | 81 | 0 | 40 |
| acid | titanate | | | | |
| Isostearic | p-toluene- | 4.5 | 99 | 10 | 95 |
| acid | sulfonic | | | | |
| | acid | | | | |
| Oleic | p-toluene- | 18.5 | 93 | 40 | 100 |
| acid | sulfonic | | | | |
| | acid | | | | |

### Example 11

### Preparation of 28-amide Derivatives of Betulin

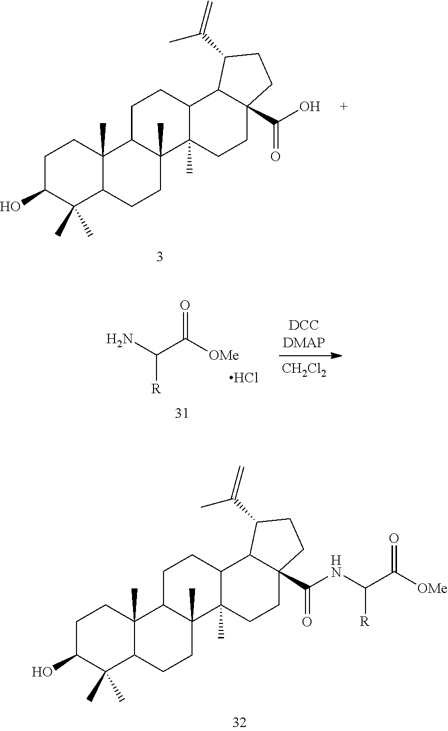

Betulinic acid 3 was prepared by oxidizing betulin 1 according to U.S. Pat. No. 6,280,778. Betulinic acid 3 (5 mmol) and aminoacid methyl ester hydrochloride 31 (5 mmol) were weighed in a flask and dissolved in dichoromethane. The flask was purged with argon, dichloromethane (5 mmol) and DMAP (2.5 mmol) were added and mixing was continued for 20 hours. The reaction mixture was diluted with ethyl acetate, washed with water, dried over sodium sulfate, and the solvent was evaporated to dryness. The betulinic acid amide 32 crude product may be purified by chromatography, if necessary. Reaction conditions and crude yields of the products are shown in Table 2.

TABLE 2

| Amino acid | Reaction time (h) | Total yield (%) |
|---|---|---|
| L-aspartate dimethyl ester, HCl | 19 | >95 |
| L-histidine methyl ester, HCl | 18 | >95 |
| L-glutaminic acid methyl ester, HCl | 19 | >95 |
| L-lysine methyl ester, HCl | 19 | >95 |

### Example 12

### Preparation of 28-aspartateamide Dimethyl Ester of Betulonic Acid

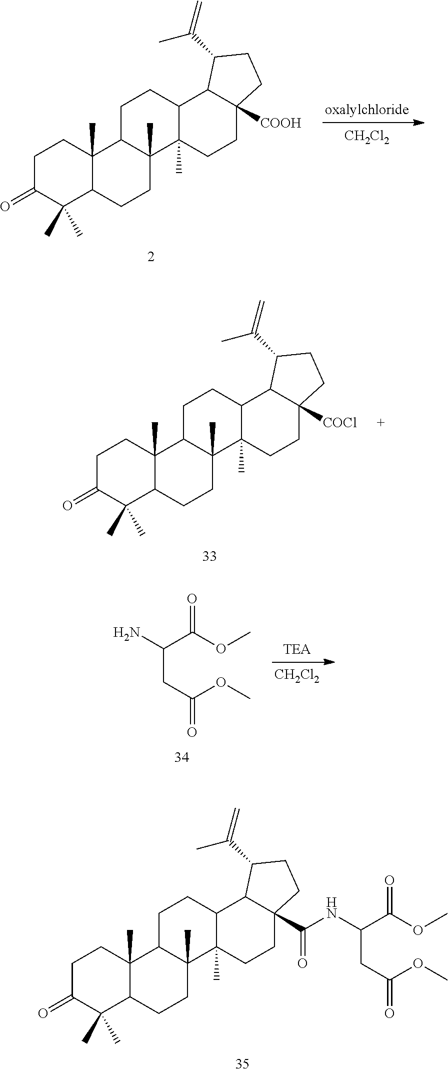

Betulonic acid 2 (8.8 mmol) was dissolved in dichloromethane under inert atmosphere, followed by the addition of oxalyl chloride (18.6 mmol) to the solution thus obtained. The reaction mixture was agitated at room temperature for 20 hours. After completion of the reaction, the solvent was evaporated to dryness and the residue was again dissolved in dichloromethane, which was once more evaporated to dryness. The crude product obtained was washed with diethyl ether. The yield was 7.5 mmol (85%) of betulonic acid chloride 33. Betulonic acid chloride 33 (4.2 mmol) and L-aspartic acid dimethyl ester hydrochloride 34 (5.5 mmol) were dissolved in dichloromethane, and triethyl amine (11 mmol) was added. The reaction mixture was agitated at room temperature for 20 hours. The reaction mixture was washed with diluted hydrochloric acid solution, water and dried over sodium sulfate. The solvent was evaporated to dryness, followed by purification of the crude product by chromatography, if necessary. Yield was 1.8 mmol (43%) of the 28-aspartateamide dimethyl ester of betulonic acid 35.

### Example 13

### Preparation of 28-N-acetylanthranilic Acid Ester of Betulin

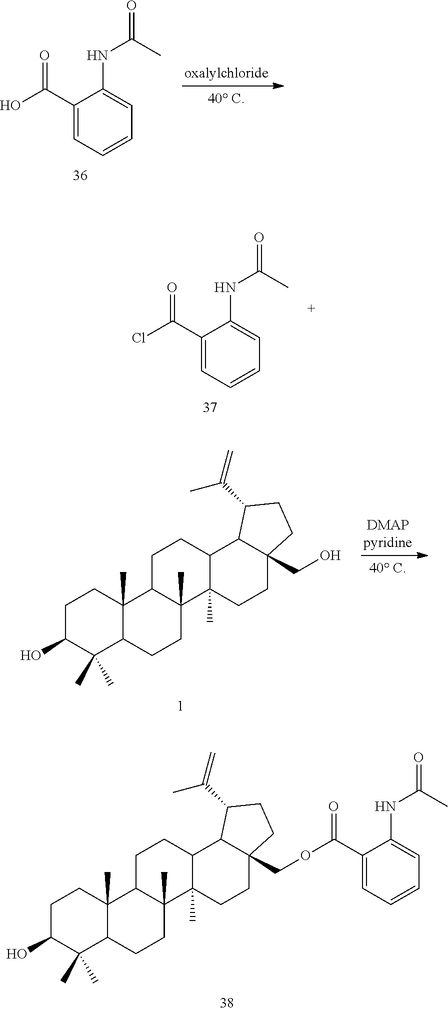

A mixture of N-acetylanthranilic acid 36 (25.0 mmol) and oxalyl chloride (250 mmol) was mixed for 16 hours at 40° C. Excessive oxalyl chloride was removed by evaporating the reaction mixture to dryness. The residue was twice dissolved in dichloromethane, which was evaporated to dryness. N-acetylanthranilic acid chloride 37 was thus obtained with a quantitative yield. A mixture of betulin 1 (11.29 mmol), DMAP (11.29 mmol), N-acetylanthranilic acid chloride 37 and pyridine (80 ml) was agitated for 24 hours at 40° C. After completion of the reaction, the reaction mixture was diluted with ethyl acetate and washed with diluted hydrochloric acid solution, and water and dried over sodium sulfate. The solvent was evaporated, followed by purification of the crude product by chromatography, thus giving 28-N-acetylanthranilic acid ester of betulin 38 with a yield of 25%.

### Example 14

### Preparation of 28-nicotinic Acid Ester of Betulin (Comparative)

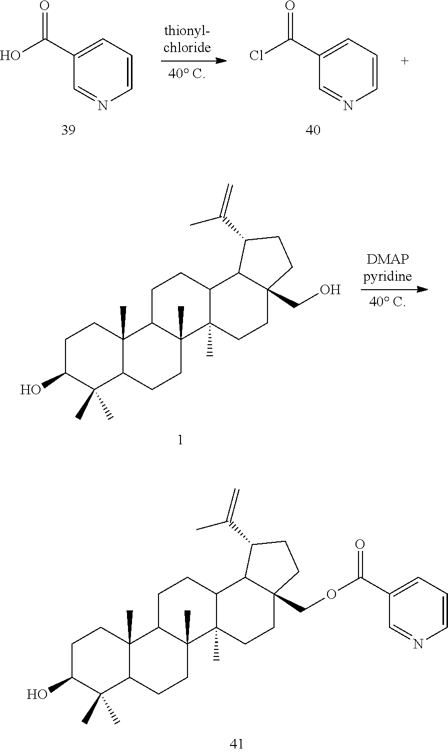

A mixture of nicotinic acid 39 (25.0 mmol) and thionyl chloride (250 mmol) was mixed for 24 h at 40° C. Excessive thionyl chloride was removed by evaporating the reaction mixture to dryness. The residue was twice dissolved in dichloromethane and evaporated to dryness. Nicotinic acid chloride 40 was obtained. A mixture of betulin 1 (2.26 mmol), DMAP (2.26 mmol), nicotinic acid chloride 40 (2.71 mmol) and pyridine (10 ml) was agitated for 24 h at 40° C. After completion of the reaction the mixture was diluted with ethyl acetate and washed with diluted hydrochloric acid solution, water and dried over sodium sulfate. The solvent was evaporated, followed by purification of the crude product by recrystallization in cyclohexane giving 28-nicotinic acid ester of betulin 41 yield 88%.

### Example 15

### Preparation 3,28-diacetoxy-19,20-ene-29-succinic Anhydride of Betulin

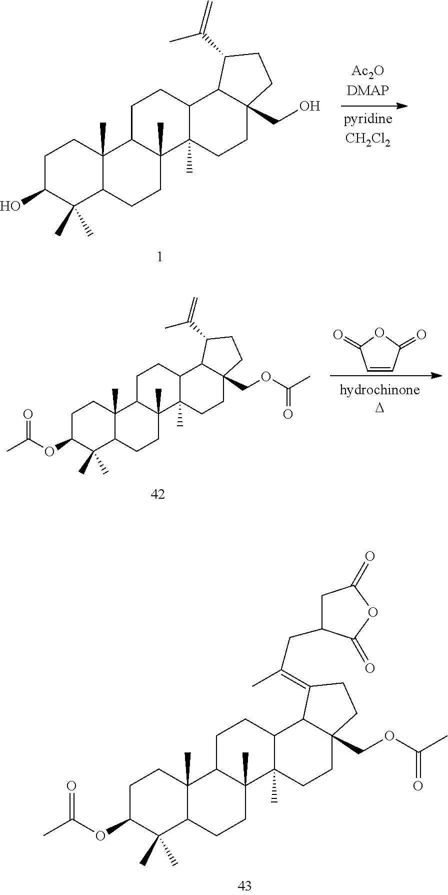

a) Acetic anhydride (19.2 ml, 203 mmol) is added to a mixture of betulin 1 (15.0 g, 33.88 mmol), DMAP (0.41 g, 3.39 mmol), pyridine (25 ml, 309 mmol), and dichloromethane (150 ml). The reaction mixture was agitated at room temperature for 17 hours. The organic phase was washed with 10% hydrochloric acid solution (200 ml), saturated NaHCO₃ solution (400 ml), water (100 ml), and dried over Na₂SO₄. The solvent was evaporated in vacuum, thus giving 3,28-diacetoxy betulin 42 with a yield of 97%.

b) A mixture of 3,28-diacetoxy betulin 42 (4.57 g, 8.68 mmol) and hydrochinone (96 mg, 0.87 mmol) was heated at 200° C., followed by the addition of succinic anhydride (2.50 g, 25.02 mmol) during 2 hours to the reaction flask. After completion of the reaction, the crude product, 3,28-diacetoxy-19,20-ene-29-succinic anhydride of betulin 43 was obtained with a yield of 100% (5.41 g, 8,65 mmol).

### Example 16

### Preparation of Betulin 3,28-dibetaine Ester

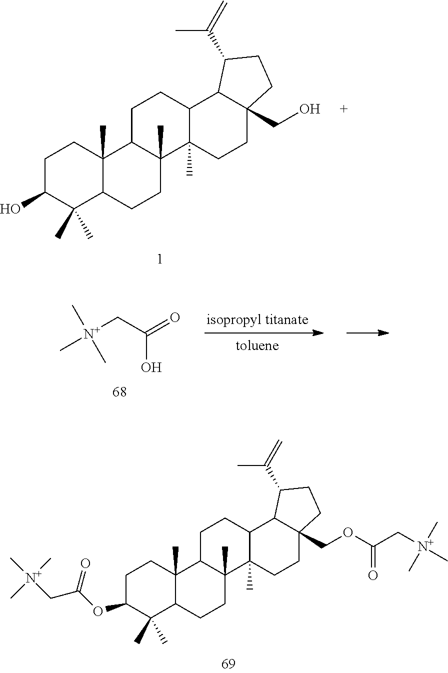

Betulin 1 (7.0 g, 16 mmol) and betaine 68 (3.8 g, 32 mmol) were dissolved in toluene (150 ml) while heating. Thereafter, isopropyl titanate Ti(OCHMe₂)₄ catalyst (0.85 g, 3 mmol) was added, and the mixture was refluxed for 3 hours. The solid final product was separated by filtration. Tetrahydrofurane was added to remove by-products, and filtering was repeated. Yield of the final product 69 (betulin 3,28-dibetaine ester) was 2.7 g (4.1 mmol, 26%).

### Example 17

### Antifeedant Activity of Compounds Derived from Betulin for Beetles of the Genus Phyllotreta spp.

Antifeedant activity for rapeseed beetles of the genus Phyllotreta spp. was tested using betulin 3,28-O-isostearylic diester (prepared as described in example 10), 3,28-diacetoxy betulin (prepared in example 15a), and betulin. Test compounds dissolved in 2 ml of rapeseed oil were sprayed on rapeseed seedlings at the cotyledon stage in an amount of 100 μg/cm². The plants thus treated were placed in an insect cage, followed by the introduction of the beetles after 24 hours. In the tests, 20 beetles were used per cage, each cage comprising 3 petridishes with seedlings, one control dish and two different test dishes, the number of the cages (=parallel tests) being 5. Pure rapeseed oil was used as the control. The beetles were allowed free access to all plants, and after one week, the test was stopped, the plants were examined, and feeding damages caused by the beetles were evaluated.

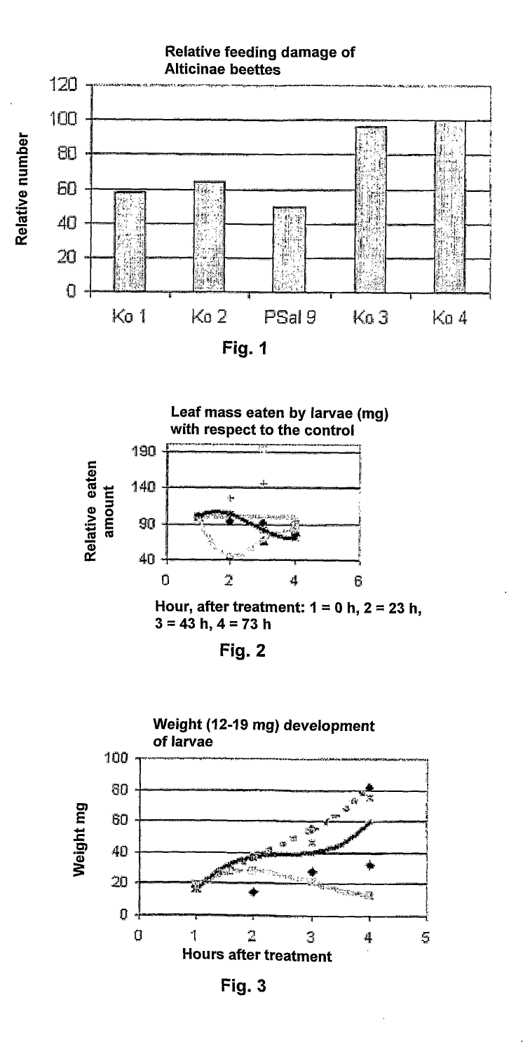

Number of the damaged leaves and the degree of damage were evaluated (% leaf surface area fed, palatability assessment). Betulin 3,28-O-isostearylic acid diester and betulin, both less complex than the control sample, efficiently prevented beetle feeding, as may be seen from the diagram shown in FIG. 1 and the palatability test in table 3 below, where rapeseed oil=100%.

Betulin 20,29-epoxy-3β,28-diacetate was used as the control compound, prepared as disclosed in J. Arg. Food Chem. 1995, 43, 2513-2516.

TABLE 3

| | % Surface area of the |
|---|---|
| Test compound | leaves eaten |
| Betulin (Ko1) | 58 |
| Betulin 3,28-O-isostearylic acid diester (Ko2) | 64 |
| Betulin 20,29-epoxy-3β,28-diacetate (Psal9) | 50 |
| 3,28-diacetoxy betulin (Ko3) | 96 |
| Rapeseed oil (Ko4) | 100 |

### Example 18

### Antifeedant Activity of Compounds Derived from Betulin for Cabbage White, Mamestra brassicae

Antifeedant activity for the cabbage white, Mamestra brassicae larvae was tested with six compounds derived from betulin. Discs cut from cabbage leaves, having a diameter of 5 cm, were placed on petridishes and treated with the test agents in an amount of 100 μg/cm². One small larva was introduced on each plate. Each agent was tested in 10 parallel studies. Cabbage leaf mass remaining on the dish was weighed after 0, 21, 43 and 73 hours after treatment (hat), and leaf mass eaten by the larva was calculated as percentage based on the control.

FIG. 2 graphically shows the leaf mass eated by the larva vs. control. Betulinic acid (lowest curve) and betulin 28-C₁₈-alkylensuccinic acid ester (curve in the middle) clearly had antifeedant activities on the larvae of cabbage white, Mamestra brassicae. It may be assumed that the activities are more distinct in choice tests (here: no-choice test).

FIG. 3 shows the weight development of the larvae (12 to 19 mg). Weight development of small larvae suggests that particularly the treatment with betulinic acid would eventually result in extermination of the larvae: Weight after 7 hours was lower than the initial weight.

Leaf mass eated by the larvae vs. control is shown in table 4.

TABLE 4

| | Time/0 h | Time/21 h | Time/43 h | Time/73 h |
|---|---|---|---|---|
| Test substance | at/% | at/% | at/% | at/% |
| 1 = control (untreated) | 100 | 100 | 100 | 100 |
| 2 = betulin | 100 | 101* | 193* | 88 |
| 3 = betulinic acid | 100 | 46 | 67 | 80 |
| 4 = betulonic acid | 100 | 94 | 95 | 86 |
| 5 = betulin | 100 | 103* | 82 | 71 |
| 28-C₁₈-alkylen- | | | | |
| succinic acid ester | | | | |
| 6 = betulin 3,28-C₁₈- | 100 | 93 | 90 | 77 |
| alkylensuccinic acid | | | | |
| diester | | | | |
| 7 = betulin 28- | 100 | 126* | 146* | 94 |
| carboxymethoxy menthol | | | | |
| ester | | | | |

*In comparison to untreated controls, higher proportion of the discs were eaten

### Example 19

### Effect of Betulin Derivatives on Feeding Behaviour of Snails

Deroceras agreste (field snails) and Deroceras reticulatum (false field snails) snails with lengts of about 2 cm, collected from nature, were used in this antifeedant test. Cabbage and lettuce leaves cut into discs with diameters of 4 cm serving as feed plants were placed on petridishes. Half on each leaf disc was treated with the test substance, the other haft serving as the control. One snail was placed on each plate, and only tests where the snail ate the leaf were assayed. The results were recorded at 36 hours after test initiation. Acetone was used as control, while betulin, betulinic acid, betulonic acid and betulin 28-carboxymethoxy mentholester dissolved in aceton served as test substances. The leaves studied were treated with test substances used in amounts of 100 μg/cm², The results from this test are presented in the following table 5.

TABLE 5

| | Percentage of the | Percentage of the leaf half |
|---|---|---|
| | control vs. | treated with the test substance |
| Test substance | total mass eaten | vs. total mass eaten |
| Betulin | 77.5 | 22.5 |
| Betulinic acid | 62.4 | 37.6 |
| Betulonic acid | 74.2 | 25.8 |
| Betulin | 63.4 | 36.6 |
| 28-carboxymethoxy | | |
| menthol ester | | |

Pure acetone had no effect on feeding of the snails, treated and untreated controls were eaten everywhere. Best results were obtained with the test substances betulinic and betulonic acids, and with betulin 28-carboxymethoxy menthol ester.

### Example 20

### Antifeedant Activity of Compounds Derived from Betulin for Strawberry Beetles (Galerucella tenella)

Antifeedant activity of betulin and betulonic acid was tested for strawberry beetles (Galerucella tenella) with adults and larvae using leaves of the plants. The test substances were dissolved in ethanol, and the leaves were treated to achieve a concentration of approx. 100 μg/cm² of the test substance. The experiment was carried out as a selection test on a petridisc containing a strawberry leaf having one half treated with the test substance and the other half treated with ethanol only. Two adult strawberry beetles (Galerucella tenella) were placed on the disc (male and female) or two larvae. The number of cavities eaten by the animals were counted daily during four days; simultaneously the number eggs laid by the adults were counted. Both the adults and the larvae had 10 tests. Results from the palatability test and the prevention of egg laying test with strawberry beetles (Galerucella tenella) are shown in the following table 6. The prevention of feeding of adults was tested only with betulonic acid, which clearly prevented feeding (approx. 41%); however the treatment prevented very strongly laying of eggs on the leaves (more than 90%). Betulin (78%) and betulonic acid (82%) both prevented feeding of larvae significantly.

TABLE 6

| | | Percentage of |
|---|---|---|
| Test substance | | the number or |
| and development | Percentage of the relative | eggs laid by the |
| stage of the beetle | number of eaten cavities | females on the leaves |
| Betulolic acid, adults | 58.7 | 8.3 |
| Ethanol, adults | 100 | 100 |
| Betulonic acid, larvae | 17.8 | — |
| Betulin, larvae | 21.9 | — |
| Ethanol, larvae | 100 | — |

### EXAMPLE 21

### Antifeedant Activity of Compounds Derived from Betulin for Lily Beetles (Lilioceris lilii)

Antifeedant activity of betulin and betulonic acid was tested for lily beetles (Lilioceris lilii) with adults and larvae using leaves of brownlily plants. The test substances were dissolved in ethanol, and the leaves were treated to achieve a concentration of approx. 100 μg/cm² of the test substance. The experiment was carried out with adults as a selection test on a petridisc containing two leaves cut from a brown lily plant. One was treated with the test substance and the other treated with ethanol only. Two adult lilybeetles (Lilioceris lilii) were placed on the disc (male and female). The leaf mass eaten by the animals was counted daily during four days by weighing the leaves; simultaneously the number eggs laid by the adults were counted. 10 parallel tests were performed. The test with larvae was a no-choise-test, where either a treated or untreated leaf of brownlily and one beetle larvae was placed on each disc. 7 parallel tests were performed. Results from the palatability test and the prevention of egglaying test with adults and larvae of lily beetles (Lilioceris lilii) are shown in the following table 7. Betulonic acid clearly prevented feeding of lily beetle adults (approx. 78%); betulin decreased feeding to a lesser extent (approx. 47%). Betulonic acid decreased very efficiently egglaying of adults on the leaves (more than 90%). Betulonic acid decreased feeding of larvae efficiently (92%), with betulin this was not observed. In a no-choise situation the larvae died on the betulonic acid treated leaves, in other test treatments they grew normally.

TABLE 7

| Test substance | Percentage of the | |
|---|---|---|
| and development | eaten relative | Percentage of the number or |
| stage of the beetle | mass of the leaf | eggs laid by the beetles |
| Betulin, adults | 40.7 | — |
| Betulolic acid, adults | 17.0 | 8.7 |
| Ethanol control, adults | 76.4 | 100 |
| Betulin, larvae | 64.2 | — |
| Betulonic acid, larvae | 5.2 | — |
| Ethanol control, larvae | 65.3 | — |

### Example 22

### Cytotoxicity Tests of the Compounds Derived from Betulin

Caco-2 cells (cell line used as a model for human intestine) were introduced in a 96 well plate in an amount of 35 000 cells (for LDH method), 45 000 cells (for WST-1 method), or 25 000 cells (for ATP method) per well. After proliferation for 24 hours, the cells were exposed to the compounds being tested for 24 hours by adding said compounds to the cultivation medium to give a concentration of 500 mM (as stock solutions in DMSO).

The influence of the compounds on the viability of the cells was measured by three different methods. Polymyxin B was used as the control. Lactate dehydrogenase (LDH) is an enzyme found in cells, and accordingly, increased amounts thereof outside cells result from cell membrane damage. The amount of LDH in the sample due to exposure was quantified by means of an enzymatic reaction using the INT (iodonitrotetrazolium) colour reagent wherein the coloured reaction product formed was determined photometrically at 490 nm. In the WST-1 method, the metabolic activity of the cells after exposure was measured using the WST-1 reagent. Metabolic activity of a cell results in the generation of a coloured product from the reagent, said product being then used to evaluate the viability of the cells by photometric measurements (absorbance at 440 nm). In the ATP method, the amount of ATP within cells decreasing rapidly due to cellular damage was measured. In the method, ATP was luminometrically quantified by means of the ATP dependent luciferase-luciferin reaction.

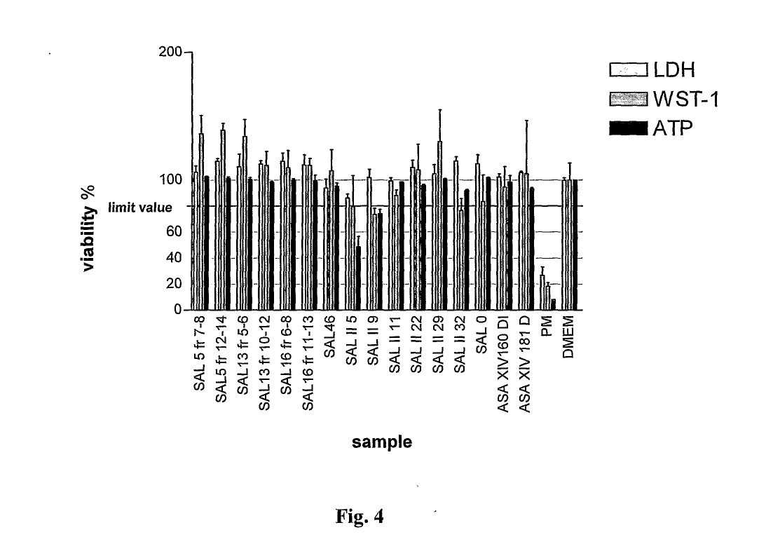

Appended FIG. 4 shows effects on the viability of Caco-2 cells (%) after exposure for 24 hour as measured by three methods for the determination of cellular viability (LDH, WSR-1 and ATP methods). Compounds exceeding the limit value, i.e. 80% viability, are considered to have no significant negative effect on the viability of cells is vitro. The compounds of the Table 8 were used for testing.

TABLE 8

| Code | Compound |
|---|---|
| PM | positive control (polymyxin B sulfate) |
| Sal-5 fr. 7-8 | 3,28-O-isostearylic acid diester of betulin |
| Sal-5 fr. 12-14 | 28-O-isostearylic acid ester of betulin |
| Sal-13 fr. 5-6 | 3,28-O-oleinylic acid diester of betulin |
| Sal-13 fr. 10-12 | 28-O-oleinylic acid ester of betulin |
| Sal-16 fr. 6-8 | 3,28-O-octanylic acid diester of betulin |
| Sal-16 fr. 11-13 | 28-O-octanylic acid ester of betulin |
| Sal-46 | 3,28-diacetoxy betulin |
| Sal-II-5 | 28-acetoxy betulin |
| Sal-II-9 | 3-oxo-28-acetoxy betulin |
| Sal-II-11 | betulinic acid |
| Sal-II-22 | 3-dehydroxybetulin |
| Sal-II-29 | 3-dehydroxy 28-acetoxy betulin |
| Sal-II-32 | betulonic acid |
| Sal-0 | betulin |
| Asa-XIV-160-DI | 28-N-acetylanthranilic acid ester of betulin |
| Asa-XIV-181-D | 28-nicotinic acid ester of betulin |

1-9. (canceled)

10. Use of compounds derived from betulin having the general formula I and salts thereof as antifeedant agents for butterflies (Lepidoptera) selected from moths (Noctuidae) belonging to the subfamily Hadeninae, daybutterflies (Rhopalocera) and littlebutterflies of subfamily Microlepidoptera, for beetles (Coleoptera) selected from subfamilies Alticinae, Galerucinae and Criocerinae, belonging to leafbeetles (Chrysomelidae), and for gastropodes (Gastropoda), where in formula I 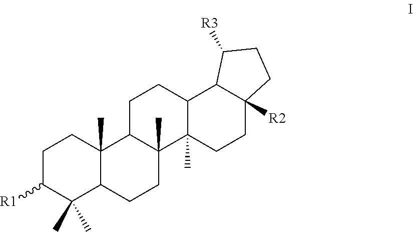
R1=OH;
R2=CH₂O(C═O)R_{f} where R_{f}=C₁₁-C₂₂ linear or branched alkyl or alkenyl group; an
R3=CH₂═CCH₃; or
R1=OH;
R2=CH₂O(C═O)CH₂(CHR_{g})COOY where R_{g}=C₄-C₂₂ linear or branched alkyl or alkenyl group, Y=H, Na, K, Ca, Mg, C₁-C₄-alkyl group, or NRₕ where Rₕ=H or C₁-C₄-alkyl group; and
R3=CH₂═CCH₃; or
R1=OH;
R2=CH₂ORᵢ where Rᵢ=2,5-diaminopentanoyl, nicotinoyl, 2-(acetylamino)benzoyl or N,N,N-trimethyl-2-oxoethanaminium group; and
R3=CH₂═CCH₃; or
R1=OH;
R2=CH₂ORₙ or CH₂O(C═O)CH₂OR′ where R′=verbenyl, terpinyl, thymyl, carvacryl, menthyl, cinnamyl, curcuminyl, eugenyl, bornyl, isobornyl group and Rₙ=chrysanthemoyl, cinnamoyl or retinoyl group; and
R3=CH₂═CCH₃; or
R1=O(C═O)Rₘ where Rₘ=C₁₁-C₂₂ linear or branched alkyl or alkenyl group;
R2=CH₂O(C═O)Rₒ where Rₒ=C₁₁-C₂₂ linear, cyclic or branched alkyl or alkenyl group; and
R3=CH₂═CCH₃; or
R1=O(C═O)CH₂(CHR_{c})COOY where R_{c}=C₄-C₂₂ linear or branched alkyl or alkenyl group, Y=H, Na, K, Ca, Mg, C₁-C₄ alkyl group or NRₕ where Rₕ=H or a C₁-C₄ alkyl group;
R2=CH₂O(C═O)CH₂(CHR_{d})COOY where R_{d}=C₄-C₂₂ linear or branched alkyl or alkenyl group, Y=H, Na, K, Ca, Mg, C₁-C₄ alkyl group or NRₖ where Rₖ=H or a C₁-C₄ alkyl group; and
R3=CH₂═CCH₃; or
R1=OR, where Rᵣ=2,5-diaminopentanoyl, nicotinoyl, 2-(acetylamino)benzoyl or N,N,N-trimethyl-2-oxoethanaminium group;
R2=CH₂ORₚ where Rₚ=2,5-diaminopentanoyl, nicotinoyl, 2-(acetylamino)benzoyl or N,N,N-trimethyl-2-oxoethanaminium group; and
R3=CH₂═CCH₃; or
R1=ORₙ or O(C═O)CH₂ORᵤ where Rᵤ=verbenyl, terpinyl, thymyl, carvacryl, menthyl, cinnamyl, curcuminyl, eugenyl, bornyl, isobornyl group and Rₙ=chrysanthemoyl, cinnamoyl or retinoyl group
R2=CH₂ORₙ or CH₂O(C═O)CH₂ORᵤ where Rᵤ=verbenyl, terpinyl, thymyl, carvacryl, menthyl, cinnamyl, curcuminyl, eugenyl, bornyl, isobornyl group and Rₙ=chrysanthemoyl, cinnamoyl or retinoyl group; and
R3=CH₂═CCH₃; or
R1=OH;
R2=(C═O)NHCHRₓCOOY where Y=H, Na, K, Ca, Mg, C₁-C₄ alkyl group or NR_{y} where R_{y}=H or a C₁-C₄ alkyl group and Rₓ=CH₂CH₂CH₂CH₂NH₂, 4-imidazolylmethyl, 3-indolylmethyl group or CH₂COOZ or CH₂CH₂COOZ group and Z=R_{y}; and
R3=CH₂═CCH₃; or
R1=oxo group;
R2=(C═O)NHCHRₓCOOY where Y=H, Na, K, Ca, Mg, C₁-C₄ alkyl group or NR_{y} where R_{y}=H or a C₁-C₄ alkyl group, and Rₓ=H, C₁-C₄-alkyl, benzyl, 4-hydroxybenzyl, CH₂CH₂CH₂CH₂NH₂, 4-imidazolylmethyl, 3-indolylmethyl or a CH₃SCH₂ or CH₂COOZ or CH₂CH₂COOZ group and Z=R_{y}; and
R3=CH₂═CCH₃; or
R1=oxo group;
R2=(C═O)OR_{w} where R_{w}=verbenyl, terpinyl, thymyl, carvacryl, menthyl, cinnamyl, curcuminyl, eugenyl, bornyl, isobornyl group; and
R3=CH₂═CCH₃
R1=OH or O—(C═O)R_{b} where R_{b}=C₁-C₂₂ alkyl or C₁-C₂₂ alkenyl group or a phenyl group;
R2=CH₂OH or CH₂O(C═O)R_{f} where R_{f}=C₁-C₂₂ alkyl or C₁-C₂₂ alkenyl group or a phenyl group; and
R3=H₂C═CCH₂R_{q} or CH₃CCH₂R_{q} where R_{q}=3-dihydrofuran-2,5-dione, 3-pyrrolidine-2,5-dione or CH(COORo)CH₂COOR_{z} where Rₒ=H, Na, K, Ca, Mg or a C₁-C₂₂ linear or branched alkyl or alkenyl group and R_{z}=H, Na, K, Ca, Mg or a C₁-C₂₂ linear or branched alkyl or alkenyl group; or
betulinic acid, betulonic acid, 3-oxo-28-acetoxybetulin, 3,28-diacetoxybetulin, 28-acetoxybetulin.

11. Use according to claim 10, characterized in that the compound derived from betulin is selected from the group consisting of 3,28-O-isostearylic acid diester of betulin, 28-O-isostearylic acid ester of betulin, 3,28-O-oleinylic acid diester of betulin, 28-O-oleinylic acid ester of betulin, 3,28-O-octanylic acid diester of betulin, 28-O-octanylic acid ester of betulin, 3-oxo-28-acetocy betulin, 3,28-diacetoxy betulin, 28-acetoxy betulin, betulinic acid, betulonic acid, 28-N-acetylanthranilic acid ester of betulin, 28-nicotinic acid ester of betulin, 28-C₁₈-alkylenesuccinic acid ester of betulin, 3,28-C₁₈-alkylenesuccinic acid diester of betulin, betulin 28-carboxymethoxy menthol, the 28-carboxymethoxy thymol ester of betulin, betulin 28-chrysanthemate, 28-cinnamic acid ester of betulin, L-aspartate amide of betulinic acid, L-histidine amide of betulinic acid, L-glutamine amide of betulinic acid, L-lysine amide of betulinic acid, and 28-aspartate amide dimethyl ester of betulonic acid.

12. Use according to claim 10 or 11, characterized in that the compound derived from betulin is applied on a base or population in an amount ranging between 1 and 1000 μg/cm², preferably between 5 and 200 μg/cm², optionally in combination with a carrier and/or medium.

13. A compound derived from betulin of the general formula I′ or a salt thereof, where in formula I′ 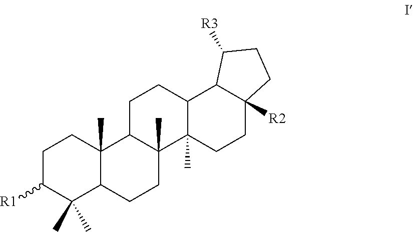
R1=OH;
R2=CH₂O(C═O)R_{f} where R_{f}=isostearyl group; and
R3=CH₂═CCH₃; or
R1=OH;
R2=CH₂O(C═O)CH₂(CHRg)COOY where R_{g}=C₄-C₂₂ linear or branched alkyl or alkenyl group, Y=H, Na, K, Ca, Mg, C₁-C₄-alkyl group, or NRₕ where Rₕ=H or C₁-C₄-alkyl group; and
R3=CH₂═CCH₃; or
R1=OH;
R2=CH₂ORᵢ where Rᵢ=2,5-diaminopentanoyl, 2-(acetylamino)benzoyl or N,N,N-trimethyl-2-oxoethanaminium group; and
R3=CH₂═CCH₃; or
R1=OH;
R2=CH₂ORₙ or CH₂O(C═O)CH₂OR′ where R′=verbenyl, terpinyl, thymyl, carvacryl, menthyl, cinnamyl, curcuminyl, eugenyl, bornyl, isobornyl group and Rₙ=retinoyl group;
and
R3=CH₂═CCH₃; or
R1=O(C═O)Rₘ where Rₘ=isostearyl group;
R2=CH₂O(C═O)Rₒ where R₀=isostearyl group; and
R3=CH₂═CCH₃; or
R1=O(C═O)CH₂(CHR_{c})COOY where R_{c}=C₄-C₂₂ linear or branched alkyl or alkenyl group, Y=H, Na, K, Ca, Mg, C₁-C₄ alkyl group or NRₕ where Rₕ=H or a C₁-C₄ alkyl group;
R2=CH₂O(C═O)CH₂(CHR_{d})COOY where R_{d}=C₄-C₂₂ linear or branched alkyl or alkenyl group, Y=H, Na, K, Ca, Mg, C₁-C₄ alkyl group or NRₖ where Rₖ=H or a C₁-C₄ alkyl group; and
R3=CH₂═CCH₃; or
R1=ORᵣ where Rᵣ=2,5-diaminopentanoyl, 2-(acetylamino)benzoyl or N,N,N-trimethyl-2-oxoethanaminium group;
R2=CH2ORₚ where Rₚ=2,5-diaminopentanoyl, 2-(acetylamino)benzoyl or N,N,N-trimethyl-2-oxoethanaminium group; and
R3=CH₂═CCH₃; or
R1=ORᵥ or O(C═O)CH₂OR′ where R′=verbenyl, terpinyl, thymyl, carvacryl, menthyl, cinnamyl, curcuminyl, eugenyl, bornyl, isobornyl group and Rᵥ=retinoyl group;
R2=CH₂ORᵤ or CH₂O(C═O)CH₂OR′ where R′=verbenyl, terpinyl, thymyl, carvacryl, menthyl, cinnamyl, curcuminyl, eugenyl, bornyl, isobornyl and Rᵤ=retinoyl group;
R3=CH₂═CCH₃; or
R1=OH;
R2=(C═O)NHCHRₓCOOY where Y=H, Na, K, Ca, Mg, C₁-C₄ alkyl group or NR_{y} where R_{y}=H or a C₁-C₄ alkyl group, and Rₓ=CH₂CH₂CH₂CH₂NH₂ or 4-imidazolylmethyl group; and
R3=CH₂═CCH₃; or
R1=oxo group;
R2=(C═O)NHCHRₓCOOY where Y=H, Na, K, Ca, Mg, C₁-C₄ alkyl group or NR_{y} where R_{y}=H or a C₁-C₄ alkyl group, and Rₓ=CH₂CH₂CH₂CH₂NH₂, 4-imidazolylmethyl or 3-indolylmethyl group or CH₂COOZ or CH₂CH₂COOZ group and Z=R_{y}; and
R3=CH₂═CCH₃; or
R1=oxo group;
R2=(C═O)OR_{w} where R_{w}=verbenyl, terpinyl, thymyl, carvacryl, menthyl, cinnamyl, curcuminyl, eugenyl, bornyl, isobornyl group; and
R3=CH₂═CCH₃; or
R1=OH or O—(C═O)R_{b} where R_{b}=C₁-C₂₂ alkyl or C₁-C₂₂ alkenyl group or a phenyl group;
R2=CH₂OH or CH₂O—(C═O)R_{f} where R_{f}=C₁-C₂₂ alkyl or C₁-C₂₂ alkenyl group or a phenyl group; and
R3=H₂C═CCH₂R_{q} or CH₃CCH₂R_{q} where R_{q}=3-dihydrofuran-2,5-dione, 3-pyrrolidine-2,5-dione or CH(COORₒCH₂COOR_{z} where Rₒ=H, Na, K, Ca, Mg or a C₁-C₂₂ linear or branched alkyl or alkenyl group and R_{z}=H, Na, K, Ca, Mg or a C₁-C₂₂ linear or branched alkyl or alkenyl group.

14. Compound derived from betulin according to claim 13, characterized in that the compound is selected from the group consisting of 28-C₁₈-alkylenesuccinic acid ester of betulin, 3,28-C₁₈-alkylenesuccinic acid diester of betulin, 3,28-O-isostearylic acid diester of betulin, 28-O-isostearylic acid ester of betulin, betulin 28-carboxymethoxy menthol, 28-carboxymethoxy thymol ester of betulin, betulin 28-chrysanthemate, L-aspartate amide of betulonic acid, L-histidine amide of betulonic acid, L-glutamine amide of betulonic acid, L-lysine amide of betulonic acid and 28-aspartate amide dimethyl ester of betulonic acid.

15. An antifeedant composition for preventing of feeding of butterflies (Lepidoptera) selected from moths (Noctuidae) belonging to subfamily Hadeninae, daybutterflies (Rhopalocera) and littlebutterfly suborder (Microlepidoptera), beetles (Coleoptera) selected from subfamilies Alticinae, Galerucinae and Criocerinae of leafbeetles (Chrysomelidae), and gastropods (Gastropoda), characterized in that the composition comprises a compound derived from betulin according to claim 13 or 14 or a salt thereof.

16. An antifeedant composition according to claim 15, characterized in that the composition comprises excipients and media selected from surface active agents, emulgators, dispersants, solvents and natural oils.
